(19) United States
(12) Patent Application Publication
(10) Pub. No.: US 2023/0364306 A1
(43) Pub. Date: Nov. 16, 2023

(54) THREE-DIMENSIONAL BODY IMPLANTS

(71) Applicants: UNIVERSITE CLAUDE BERNARD LYON 1, Villeurbanne (FR); CENTRE NATIONAL DE LA RECHERCHE SCIENTIFIQUE (CNRS), Paris (FR); ECOLE CENTRALE DE NANTES, Nantes (FR); ECOLE SUP CHIMIE PHYS ELECTRONIQ LYON, Villeurbanne CEDEX (FR); HEALSHAPE, Villeurbanne (FR)

(72) Inventors: Christophe MARQUETTE, Villeurbanne (FR); Audrey CHERBLANC, Sarcey (FR); Morgan DOS SANTOS, Saint Verand (FR); Luciano VIDAL, Nantes (FR); Emma PETIOT, Villeurbanne (FR); Laura CHASTAGNIER, Lyon (FR); Amélie THEPOT, Lyon (FR); Baptiste GODET, Perros Guirec (FR)

(73) Assignees: UNIVERSITE CLAUDE BERNARD LYON 1, Villeurbanne (FR); CENTRE NATIONAL DE LA RECHERCHE SCIENTIFIQUE (CNRS), Paris (FR); ECOLE CENTRALE DE NANTES, Nantes (FR); ECOLE SUP CHIMIE PHYS ELECTRONIQ LYON, Villeurbanne CEDEX (FR); HEALSHAPE, Villeurbanne (FR)

(21) Appl. No.: 18/029,290

(22) PCT Filed: Jun. 24, 2022
(86) PCT No.: PCT/FR2022/051265

§ 371 Date: Mar. 29, 2023

(30) Foreign Application Priority Data
Jun. 25, 2021 (FR) FR2106827

(51) Int. Cl. A61L 27/52; A61L 27/26; A61L 27/56; B33Y 80/00; B33Y 40/20; B33Y 10/00; B29C 64/30; B29C 71/00; B29C 64/118

(52) U.S. Cl. CPC A61L 27/52; A61L 27/26; A61L 27/56; B33Y 80/00; B33Y 40/20; B33Y 10/00; B29C 64/30; B29C 71/0009; B29C 64/118; A61L 2430/04; B29K 2089/00; B29K 2005/00; B29K 2995/0077; B29L 2031/7532

(57) ABSTRACT

Three-dimensional body implants including a hydrogel, which includes cross-linked alginate and gelatin, and in particular breast implants. The hydrogel of the implants has a mechanical strength of 1 kPa to 1000 kPa, and the hydrogel of the implants may further include fibrinogen. The implants include a porous zone, and the implants are acellular, i.e., free of cells during their manufacture.

### FIELD OF THE INVENTION

The present invention relates to the general field of bio-materials and in particular to implants, intended to be introduced into the human body/living organism, in particular to replace and/or augment a more or less soft tissue and/or to fill a space between the skeleton and the skin, or sutured onto the skin.

The present invention relates to three-dimensional body implants comprising a hydrogel which comprises cross-linked alginate and gelatin, and in particular to temporary or permanent implants. The implants of the invention have a determined and particularly advantageous porosity and mechanical strength. These implants are also acellular, i.e. no cells, and in particular no living cells, are incorporated into the implant during its manufacture. In all aspects of this invention, the hydrogel may further comprise fibrinogen.

### STATE OF THE ART

Hydrogel-based structures comprising alginate and gelatin are known from the state of the art, but they lack satisfactory mechanical strength, because these constituents usually have limited elasticity (low Young's modulus, in particular), which makes the resulting structures difficult to manipulate.

Recent scientific reviews by Armin Vedadghavami et al., 2017, Acta Biomaterialia 62, 42-63, Marta Calvo Catoira et al., 2019, Journal of Materials Science: Materials in Medicine, 30:115, and Gils Jose et al., 2020, Current Medicinal Chemistry, 27, 2734-2776, highlight the biocompatible properties of natural hydrogels, especially those based on alginate and gelatin, but also their limitations in terms of mechanical properties. Indeed, these natural polymers have mechanical resistances that are too low for the production of manipulable and/or implantable devices and for the implementation of products with complex structures and dimensions larger than 5 cm³, thus limiting the clinical applications of this technology. The mechanical properties of the structures obtained in the prior art remain insufficient to make them suitable for manipulation. Moreover, in the case of structures intended to be implanted in the human or animal body, and if necessary sutured, there is a need to obtain structures which have adequate mechanical properties, and whose degradability in contact with living cells or tissues is not too rapid.

To achieve regenerative and resorbable implants, a structure with a macroscopic pore size (e.g. greater than 100 μm) is very favorable. Furthermore, this type of medical application often requires large volume implants.

To date, the literature does not describe any macroporous implant, in particular of large volume, composed of a hydrogel since the fabrication of large and porous objects from hydrogel is limited by the weak mechanical properties of hydrogels.

### SUMMARY

One of the aims of the invention is to overcome the disadvantages of the implants of the prior art, and to make it possible to provide biocompatible implants, whose main constituents are of natural origin.

Indeed, the implants of the invention have particularly advantageous and innovative characteristics, especially in terms of (i) the mechanical strength of the constituents, which is similar to that of native tissues when they are introduced, (ii) stability over time, (iii) flexibility, (iv) remarkable resistance to tearing and impact, and (v) colonization by the cells of the host organism.

According to a first aspect, the invention provides a three-dimensional body implant which comprises a hydrogel comprising cross-linked gelatin and cross-linked alginate, wherein said hydrogel has a mechanical strength of 1 kPa to 1000 kPa and wherein said implant has at least one porous zone, the porous zone comprising a plurality of pores each having a pore size, the porous zone having an overall porosity between 100 μm and 10,000 μm, the overall porosity corresponding to an average of the pore sizes measured in the porous zone.

The pores of the porous zone may have homogeneous pore sizes, i.e., differing from each other by no more than 15%.

The pores of the porous zone may be homogeneously, i.e., evenly distributed.

The pores of the porous zone may extend along central axes having respectively homogeneous orientations, i.e. differing from each other by not more than 20°.

The central axes of the pores of the porous zone can be arranged with homogeneous spacings, i.e., not differing from one another by more than 15%.

The pores of the porous zone may each have homogeneous geometries, i.e., the contours of which may be superimposed with more than 50% of the portions overlapping or being parallel.

The pores of the porous zone can be separated from each other by material strands having respectively homogeneous thicknesses, i.e. not differing from each other by more than 15%.

The gelatin may be cross-linked by an enzyme, preferably a transglutaminase.

The implant may include a plurality of porous zones.

Said plurality of porous zones may include at least two porous zones in which the pores have different pore sizes and/or shapes.

The porous zones may be arranged to form a gradient of pore sizes distributed across the implant, with the porous zones following one another along a gradient direction in an order selected from an ascending order and a descending order of pore sizes.

The implant may include:
a first porous zone forming a base representing 5% to 40%, preferably 20% to 40%, of a total volume of the implant, and having a pore size between 500 micrometers and 5000 micrometers, particularly 250 micrometers to 800 micrometers,
a second porous zone forming a core representing 20% to 70%, preferably 30% to 50%, of the total volume of the implant and having a pore size between 500 micrometers and 2500 micrometers, in particular 100 micrometers to 250 micrometers
a third porous zone forming a shell representing 5% to 40%, preferably 10% to 40%, of the total volume of the implant, and having a pore size between 1000 micrometers to 10000 micrometers, in particular 1000 micrometers to 2500 micrometers.

The implant may include at least one non-porous zone, the non-porous zone having a fill rate greater than 99%.

The at least one non-porous region may include a perimeter surrounding the porous region.

Said at least one porous zone may cover a substantial portion of the implant, i.e., at least 50%, preferably at least 75%, particularly at least 90%, for example at least 95%.

The implant may consist of a plurality of layers each having a mesh consisting of a plurality of meshes, the layers being stacked on top of each other such that the meshes form the pores.

The meshes of each layer may have homogeneous mesh sizes, i.e., differing by no more than 15% from one another.

The meshes in each layer may be homogeneously, i.e., evenly distributed.

The meshes of each layer may extend around central mesh axes having respectively homogeneous orientations, i.e. not differing from each other by more than 20°.

The central mesh axes of the meshes of each layer can be arranged with homogeneous spacings, i.e. not differing from each other by more than 15%.

The meshes of each layer may have homogeneous geometries, i.e., the contours of which may be superimposed with more than 50% of the portions overlapping or being parallel.

The meshes of each layer can be separated from each other by material strands each having homogeneous thicknesses, i.e. not differing from each other by more than 15%.

The implant may have a volume in the range of 0.05 mL to 3 L, preferably 100 mL to 600 mL.

The implant may be a breast implant.

According to another aspect, the invention proposes a three-dimensional body implant, in particular as defined above, obtainable by a manufacturing process comprising successively:
a step of preparing a hydrogel comprising gelatin and alginate,
a step of three-dimensionally shaping the hydrogel so as to form at least one porous zone, the porous zone comprising a plurality of pores each having a pore size, the porous zone having an overall porosity of between 100 μm and 10000 μm, the overall porosity corresponding to an average of the pore sizes measured in the porous zone, and
a step of cross-linking the hydrogel with at least one divalent cation, preferably calcium, and transglutaminase, said hydrogel having a mechanical strength of 1 kPa to 1000 kPa.

During the cross-linking step, the divalent cation and transglutaminase may be added concomitantly.

The hydrogel may comprise from 0.5% to 3% alginate and from 1% to 17.5% gelatin.

The hydrogel may further comprise cross-linked fibrinogen, and preferably up to 2% cross-linked fibrinogen.

The manufacturing process may further provide for using thrombin during the cross-linking step.

During the three-dimensional shaping step, the manufacturing process may provide for implementing an additive manufacturing process, particularly 3D printing.

The manufacturing process may further comprise a sterilization step.

According to another aspect, the invention relates to a method for implementing the implant as defined above in the context of reconstructive or cosmetic surgery, comprising a step of implanting the implant, in particular a breast implant, in the body of a subject, in particular the breast of a subject.

### DEFINITIONS

In this invention, the terms below are defined as follows:
“Cross-linking agent”, in the context of the present invention, means an agent capable of cross-linking hydrogel components, in particular alginate, gelatin and fibrinogen.
“Biodegradable” means the ability to be destroyed by a living organism. In particular when the implant is implanted in the host, said implant is biodegradable if it is capable of being destroyed by said host.
“Fibers”, in the context of the present invention, refers to any element of filamentary appearance, generally occurring in bundles.
“Gradient”, in the context of the present invention, means the gradual evolution from one pore size to another in an increasing or decreasing manner
“Host” and “recipient”, in the context of the present invention, are equivalent terms and used interchangeably to refer to the organism into which the implants according to the invention may be introduced.
“Shaping”, in the context of the present invention, consists in giving the hydrogel a particular shape and structure or architecture, and in particular adapted to the destination of the hydrogel once consolidated.
“Overall porosity” or “overall pore size”, in the context of the present invention, refers to the average of the pore size values measured over the porous zone(s) of the implant. It does not refer to the porosity of the hydrogel itself.
“Pore size”: in the context of the present invention, refers to the largest distance between two opposing beads of material.

### DETAILED DESCRIPTION

The present invention makes it possible to provide three-dimensional body implants, which have particularly advantageous mechanical characteristics, especially in terms of the mechanical strength of the constituents, which is similar to that of native tissues, stability over time and remarkable resistance to tearing and impact.

The implants according to the present invention can be used in place of (replacement of all or part of) or in addition to (augmentation of) various organs or tissues of the animal body and more particularly of the human body, either permanently or temporarily. By definition, the implants according to the invention are suitable for contact with living fluids or living tissues. In particular, they are intended to be implanted under the skin, or even on the skin, especially for skin regeneration and/or healing.

The implants of the invention are therefore substitutes or additions, replacing and/or increasing and/or reinforcing soft or flexible, and sometimes elastic, tissues. They are preferably used to replace entirely or partially, to increase or to reinforce connective tissues, skin and adipose tissues.

The implants according to the invention are therefore intended for plastic, reconstructive or regenerative surgery.

All the constituents of the implants of the invention described below must meet the regulatory requirements specific to devices intended to be implanted, particularly with respect to purity grades.

The implants according to the invention are therefore body implants, such as, for example, breast implants, pectoral implants, gluteal implants, facial implants or any other implant for making up for loss of tissue volume.

According to a particularly preferred embodiment, the implants of the present invention are breast implants.

The implants of the invention can be large, both in volume since they can in particular reach a volume of 0.05 mL to 3 L, and preferably 100 mL to 600 mL, but also in size, which can range from 0.5×0.5×0.2 to 20×15×15, i.e., a size within the following ranges of length×width×thickness: length of 0.5 cm to 20 cm×width of 0.5 cm to 15 cm×thickness of 0.2 cm to 15 cm. For example, the size of implants for tissue filling is generally not more than 20×15×15. It is preferably of the order of 12×12×3 or 12×12×4 for a breast implant.

Thus, according to one embodiment, the implants of the invention have a volume greater than 0.05 mL, and preferably a volume of 0.05 mL to 3 L.

The implants may be of any shape associated with the volumes mentioned in this description. For example, the implants may be in the form of half-spheres, half-drops, or any other shape that may be customized to the subject matter.

In one embodiment, the implants according to the invention are temporary because they are resorbable due to their composition, and disappear over time after their implantation in the body, leaving in their place cells and neo-tissues naturally vascularized by the host living organism. These temporary implants are therefore more precisely those that can be colonized by the cells of the host organism, in particular due to the existence of a determined porosity (using a maximum value) and/or the use of a constituent material of the implant that preserves cellular viability and is conducive to cellular proliferation.

These implants therefore define an internal space, a sort of skeleton/framework/backbone/matrix (or “scaffold” in English) allowing colonization by cells, in particular the cells of the recipient organism.

In one embodiment, the implants according to the invention are biodegradable due to their composition, and are therefore suitable for implantation in the animal body, in particular, the human body.

In one embodiment, the implants according to the invention comprise at least one porous zone.

In one embodiment, said at least one porous zone, i.e., the porous zone when there is only one or all of the porous zones when there are several, represents, by volume, about 5% to 100% of the implant, preferably about 50% to 100% of the implant, more preferably, about 90% to 100% of the implant. According to one embodiment, the porous zone or all of the porous zones comprise more than 90% of the implant. According to one embodiment, the porous zone or all of the porous zones comprise about 90%, 91%, 92%, 93%, 94%, 95%, 96%, 97%, 98%, 99% or 100% of the implant.

In one embodiment, the porous zone or all of the porous zones comprise the entire implant.

The porosity of the porous zone of the implant is a key parameter to be adjusted according to the tissues or organs concerned which are in particular to be replaced and/or increased. Indeed, the porosity translates the empty space present in the porous zone of the implant which can be adapted in order to bring more or less material and thus to confer a certain mechanical resistance which is as close as possible to that of the native tissue of the implantation zone.

The overall porosity corresponds to an average of the pore sizes measured on each porous zone.

In the context of the present invention, the implants are characterized at the level of their structure by the porosity of the porous zone, expressed here in two different but correlated and therefore equivalent or alternative ways: the pore size expressed in micrometers and/or the hydrogel filling rate expressed as a percentage (volume of hydrogel/total volume of the implant).

According to one embodiment, the porous zone comprises a plurality of pores each having a pore size.

In one embodiment, for soft tissue, a large overall pore size in the porous zone of the implant, in the range of 1000 μm to 10000 μm, in particular 1000 μm to 5000 μm, and/or a filling ratio of the porous zone of the implant, of 5% to 50% will be preferred, as the resulting implant, containing less material, will be more flexible. Preferably, the porous zone of the implant will have a fill rate of 5% to 50%, and even more preferably, 15% to 50%.

In one embodiment, for rigid tissue, a low overall pore size in the porous zone of the implant, in particular less than 1000 μm, and/or a filling ratio of the porous zone of the implant, from 50% to 99%, in particular from 50% to 95%, will be preferred as it will provide said implant with a high mechanical strength for rigid tissue. Preferably, the porous zone of the implant will have a fill rate of 50% to 99%, and even more preferably, 50% to 90%.

In addition, the pore size can be tailored to the different cell types present in the tissue. Again, a dense and low-porosity environment, with a pore size of the implant, in particular smaller than 1000 μm and/or a high filling rate of the implant, from 50% to 99%, and in particular from 50% to 95%, will be preferred for osteoblast-type cells evolving in a very rigid matrix, whereas a flexible and more porous environment, with a pore size of the implant, located in particular between 1000 μm and 5000 μm and/or a filling rate of the implant, of 5% to 50%, will favour the survival, proliferation and metabolism of fibroblast and adipocyte type cells evolving in a flexible matrix.

Finally, the choice of the pore size makes it possible to adjust the degradation time of the implant in the body. An implant with small pore size, in particular smaller than 1000 μm, and/or with a filling rate of 50% to 99%, in particular 50% to 95%, will be composed of more material, the total degradation will then be more or less long depending on the implant size. On the other hand, the more rapid the degradation of the implant is desired, for example less than 12 months, the more pores of large size are preferred in the implant, in particular between 1000 μm and 5000 μm and/or a filling rate of the implant of 5% to 50%.

When the implants according to the invention are prepared using a 3D printing technique, the pore sizes as disclosed above correspond to the length between the deposited hydrogel filaments, and in particular to the void distance between these filaments.

Thus, the present disclosure relates to a three-dimensional body implant which comprises a hydrogel comprising cross-linked alginate and cross-linked gelatin, characterized in that said hydrogel has a mechanical strength of from 1 kPa to 1000 kPa and that said implant has at least one porous zone, the porous zone comprising a plurality of pores each having a pore size, the porous zone having an overall porosity of at most 5000 μm.

The present invention also relates to a three-dimensional body implant which comprises a hydrogel comprising cross-linked alginate and cross-linked gelatin, characterized in that said hydrogel has a mechanical strength of from 1 kPa to 1000 kPa and that said implant has at least one porous zone, the porous zone comprising a plurality of pores each having a pore size, the porous zone having an overall porosity of between 100 μm and 10000 μm, in particular of at most 5000 μm, the overall porosity corresponding to an average of the pore sizes measured on the porous zone.

The hydrogel has a mechanical strength of 1 kPa to 1000 kPa.

The implants according to the invention, in view of their constituents and their structure, preferably have an apparent mechanical strength of 10 kPa to 800 kPa, even more preferably 10 kPa to 300 kPa, or even more preferably 50 kPa to 300 kPa.

The implants of the invention therefore have mechanical properties that are similar to those of native tissues that are sought to be replaced or increased.

As examples, the average mechanical strengths of different native tissues are mentioned in Table 1 below (as described by Guimarãdes C. et al. , Nature Reviews Materials volume 5, pages 351-370 (2020)):

TABLE 1

| Human tissue | Young's Modulus (kPa) |
|---|---|
| Cartilage | 1000 |
| Cornea | 300 |
| Bladder | 200 |
| Skin | 100 |
| Glandular breast tissue | 35 |
| Lung | 10 |
| Adipose tissue | 10 |
| Kidney | 8 |
| Liver | 5 |
| Brain | 1 |

The mechanical strength discussed here can also be referred to as elasticity or Young's modulus. By elasticity or Young's modulus, we mean the longitudinal modulus of elasticity or tensile modulus, which is the constant that connects the tensile (or compressive) stress and the beginning of the deformation of an isotropic elastic material.

Young's modulus is the mechanical stress that causes an elongation of 100% of the initial length of a material, that is to say a doubling of its length.

This Young's modulus is governed by Hooke's law: σ=E ε, where:
σ is the mechanical stress (in units of pressure) ;
E is Young's modulus (in unit of pressure) ;
ε is the relative elongation or strain (dimensionless); (ε=l−l₀/l₀; l₀ being the initial length and t being the one after deformation).

In addition to the singular mechanical strength imparted by their constituents, the implants of the present invention possess at least one porous zone comprising a multitude of pores each having a pore size.

In one embodiment, the porous zone has an overall porosity of at most 10000 μm. According to an embodiment, the porous zone has an overall porosity of at most 5000 μm.

The porous zone may have an overall porosity of at least 10 micrometers, for example at least 50 micrometers. According to an embodiment, the porous zone has an overall porosity of at least 100 micrometers, and even more preferably at least 500 micrometers.

Thus, according to one embodiment, the overall porosity of the porous zone of the implants according to the invention can range from 10 micrometers to 1000 micrometers, or from 20 micrometers to 1000 micrometers, or from 1000 micrometers to 5000 micrometers.

Thus, according to one embodiment, the overall porosity of the porous zone of the implants according to the invention may range from 100 micrometers to 10000 micrometers, preferably from 500 micrometers to 2500 micrometers, more preferably from 500 micrometers to 1000 micrometers or from 1000 micrometers to 2500 micrometers or from 2500 micrometers to 5000 micrometers.

In this respect, the implants according to the invention may have a plurality of porous zones, said porous zones may have different pore sizes within their three-dimensional structure, for example in the form of a porosity gradient distributed over the implant. The porous zones then follow one another along a gradient direction in an order selected from an ascending order and a descending order of pore sizes. A pore size gradient allows, for example, a selection of cell types colonizing the implant.

In one embodiment, the implants according to the present invention comprise a plurality of porous zones. In one embodiment, the implants according to the present invention comprise at least two porous zones, preferably three porous zones, of different pore sizes, for example in the form of a porosity gradient, each porous zone having a defined overall pore size. This allows, for example, to define more or less rigid zones depending on the type of tissue to be regenerated or in contact with the implant in the host organism. The porous zones can also comprise different pore shapes.

In one embodiment, the pore sizes of said porous zones preferably range from 100 micrometers to 7000 micrometers, especially from 100 micrometers to 3000 micrometers.

To produce an implant having different pore size zones, for example in the form of a porosity gradient, these zones can be defined as each having a pore size sub-range, as long as the pore size obtained in all of the porous zones remains in a range from 100 micrometers to 10000 micrometers, and preferably from 100 micrometers to 3000 micrometers.

In one embodiment, the pore size sub-ranges are preferably in the range of 100 micrometers to 250 micrometers, 250 micrometers to 800 micrometers, and 1000 micrometers to 2500 micrometers when the implant contains three different pore size zones or in the range of 100 micrometers to 250 micrometers and 250 micrometers to 3000 micrometers when the implant contains only two different pore size zones. According to one embodiment, these sub-ranges constitute a gradient of 100 micrometers to 3000 micrometers.

In one embodiment, the pore size sub-ranges are preferably in the range of 500 micrometers to 2500 micrometers, 500 micrometers to 5000 micrometers and 1000 micrometers to 10000 micrometers when the implant contains three different pore size zones or in the range of 500 micrometers to 5000 micrometers and 1000 micrometers to 10000 micrometers when the implant contains only two different pore size zones.

The architecture of the implants according to the invention can be broken down into 3 distinct zones.

A base of the implant (5% to 40% of the total volume of the implant, preferably 20% to 40%) is preferably placed directly in contact with the muscle tissue, and has an intermediate pore size (500 micrometers to 5000 micrometers, in particular 250 micrometers to 800 micrometers) favoring colonization by endothelial cells and surrounding vascular structures. Endothelial cells will readily migrate through this pore size and organize themselves into vascular/microvascular structures allowing for neovascularization of the implant and thus better integration with adjacent tissues. The easy vascularization of the structure also limits the risk of necrosis of the tissues that have colonized the implant.

A core of the implant (20% to 70% of the total implant volume, preferably 30% to 50%) is not in direct contact with the host tissues of the implantation zone. This zone has a fine pore size (500 micrometers to 2500 micrometers, especially 100 micrometers to 250 micrometers), and has a role in supporting tissue regeneration. This area is composed of more material than the other areas, so biodegradation in the body will be slower to provide the cells with a support matrix to proliferate.

A shell of the implant (5% to 40% of the total volume of the implant, preferably 10% to 40%) is preferably placed so that it is the first part in contact in case of shocks and/or compressive stresses. The shell has a large pore size (1000 micrometers to 10000 micrometers, in particular 1000 micrometers to 2500 micrometers) allowing easy migration of the cells towards the core of the implant. This shell acts as a mechanical protection for the implant core.

The pore size sub-ranges can be combined to form a gradient with a pore size in all the porous zones ranging from 100 micrometers to 10000 micrometers. The gradient preferably ranges from 500 μm to 7000 μm.

When the implants of the invention can be prepared using an additive manufacturing technique, in particular 3D printing, in particular by extrusion of viscoelastic material, the existence of pores in the implants can then be associated with a three-dimensional structure in the form of “lattices” preferably gyroid, cubic or hexagonal, whose size in the XY plane is given by the pore size and the height by the diameter of the printing filament, and in particular from 200 micrometers to 1500 micrometers, preferably from 200 micrometers to 1000 micrometers.

Thus, according to one embodiment, the pores in the porous zone(s) have a gyroid, cubic or planar hexagonal shape. According to an embodiment, the pores of the porous zone(s) have an identical shape to each other within each porous zone.

The pores in each of the porous zones may have homogeneous pore sizes, i.e., differing from each other by no more than 15%.

In one embodiment, the pores are homogeneously, evenly distributed, i.e., located equidistant from each other, throughout the volume of the porous zone(s).

More particularly, the pores of the porous zone may extend along central axes having respectively homogeneous orientations, i.e. not differing from each other by more than 20°. The central axes of the pores of the porous zone can be arranged with homogeneous spacings, i.e., not differing from each other by more than 15%.

In addition, the pores of the porous zone may each have homogeneous geometries, i.e., the contours of which may be superimposed with more than 50% of the portions merging or being parallel.

The pores of the porous zone can be separated from each other by material strands each having homogeneous thicknesses, i.e. not differing from each other by more than 15%.

In a porous zone of defined pore size and shape, the organization of the pores is characterized by the repetition of a same pattern, a pattern being composed of one or more meshes, via the translation of this same pattern along at least one direction of space.

In one embodiment, the implants according to the invention further comprise one or more non-porous zones, known as solid zones.

In one embodiment, the solid zones are zones having a filling rate greater than 99%, in particular 100% (pore size of 0 μm), which can in particular be obtained by using a manufacturing technique such as molding for example.

In one embodiment, the non-porous zone represents, by volume, about 0% to 50% of the implant, preferably about 0% to 25% of the implant, more preferably about 0% to 10% of the implant. According to an embodiment, the non-porous zone represents less than 10% of the implant. According to one embodiment, the non-porous zone is 0%, 1%, 2%, 3%, 4%, 5%, 6%, 7%, 8%, 9% or 10% of the implant.

One or more perimeters (solid structure surrounding the entire periphery of the implant) may also be present in the structure in one or more layers of thickness. This addition limits the irritation and inflammation phenomena in the body that may occur in the event of crumbling of the edges of the implant.

Solid areas forming channels passing through the implant may also be present in the structure in order to confer additional mechanical resistance to the implant. These channels act as a mechanical reinforcement of the structure. In the context of breast reconstruction, these channels are largely inspired by Cooper's ligaments in a biomimetic perspective.

The porosity and the particular structure of the implant thus allow the vascularization of the neoformed and/or grafted tissues, favors the diffusion of nutrients and metabolites, provides a support and a mechanical environment adapted to the cells, thus creating an environment favorable to the colonization and the tissue regeneration by limiting the phenomena of ischemia and necrosis of the neoformed and/or grafted tissues

The implant can also include a void zone, i.e. a volume with a filling rate equal to 0. According to an embodiment, the void zone represents, by volume, about 0% to 25% of the implant, preferably about 0% to 10% of the implant.

According to one embodiment, this void zone allows the injection of cells from the subject during the implantation of the implant in said subject. These cells will thus be able to colonize the implant.

In one embodiment, the present invention relates to an implant, in particular a breast implant, which includes a porous zone. According to one embodiment, said porous zone represents the entire implant.

In one embodiment, the present invention relates to an implant, in particular a breast implant, which comprises a porous zone and a non-porous zone, such as, for example, a perimeter as defined above. According to one embodiment, said porous zone represents, by volume, more than 90% of the implant. According to an embodiment, said non-porous zone by volume, is less than 10% of the implant.

In one embodiment, the present invention relates to an implant, in particular a breast implant, which comprises two porous zones. In one embodiment, the present invention relates to an implant, in particular a breast implant, which comprises three porous zones. According to an embodiment, said implant further comprises a non-porous zone, such as, for example, a perimeter as defined above.

In one embodiment, the present invention relates to an implant, and in particular a breast implant, which has different pore sizes distributed over three zones, for example in the form of a porosity gradient, as mentioned below in Table 2 and illustrated in FIG. 1.

TABLE 2

| Zone | Pore size (micrometer) |
|---|---|
| (1) | 1000-10000, in particular 1000-2500 |
| (2) | 500-2,500, in particular 100-250 |
| (3) | 500-5000, in particular 250-800 |
| (4) | No pore size, full zone |

The implants according to the present invention have particular advantages in the context of breast reconstruction, since the implants must be sufficiently resistant to withstand high compressive stresses in this anatomical area which is very regularly subjected to this type of stress. Because of their mechanical characteristics, in particular elasticity and flexibility, the implants of the invention make it possible to produce less mechanical stress on the host tissues in direct contact, and thus to reduce inflammation phenomena.

Various methods well known to the skilled person can be used to measure pore size. Among them, we can mention optical microscopy and electron microscopy. The void volume of the implant (inverse of the filling rate) can be measured by weighing (using the density of the material), volume displacement (Archimedes method), etc.

Preferably in the context of the present invention, the ranges disclosed herein correspond to a pore size measurement by optical microscopy. Thus, according to one embodiment, the porosity or pore size is measured by optical microscopy. According to an embodiment, the porosity or pore size is measured by electron microscopy.

The porosity characteristic of the porous zones of the implants of the invention can also be expressed by the filling ratio of the hydrogel implant structure, since varying the filling ratio can affect the porosity of the implants and vice versa. This filling ratio can, for example, be obtained by measuring the volume of the implant and measuring the void volume.

As shown in the examples, the chosen filling parameters allow to obtain a given range of pore sizes. Conversely, a given range of pore sizes correlates with particular filling parameters.

Implants of the invention can have a hydrogel fill rate ranging from 5% to 99% of the total volume of the implant. The less the implant is filled (less than 50% filling), the more flexible it will be, allowing the flexibility/firmness ratio to be adjusted, depending on its destination in the body. Conversely, the more the implant is filled (filling rate greater than 50%), the more rigid it will be.

The porosity of the implants of the invention favors colonization by cells. The three-dimensional structure, in combination with a variable pore size or filling ratio, thus makes it possible to obtain implants containing multiple cavities, which can be colonized, once the implant is in place, by cells/tissues of the host organism which can then proliferate and differentiate in situ.

The three-dimensional structure as well as the particularly advantageous mechanical properties of the implants according to the invention are maintained after sterilization, and in particular by irradiation or plasma.

Various methods well known to the person skilled in the art can be used to measure the mechanical strength of the implants, such as dynamic mechanical analysis (DMA) or compression, tensile and/or flexural tests. Examples of methods for measuring the mechanical strength of implants are described in the examples.

In one embodiment, the mechanical strength of the implants is measured by dynamic mechanical analysis (DMA). According to an embodiment, the mechanical strength of the implants is measured by compression, tensile and/or bending tests.

As mentioned, the main constituents of the implants according to the invention are of natural origin.

Alginate is a linear polysaccharide extracted from marine algae, mainly from the brown algae species Phaeophyceae. This biocompatible polymer is composed of homopolymeric blocks of 1,4β-D mannuronic acid (M) and its epimer C-5 α-L guluronic acid (G). This biopolymer consists of M-block, G-block sequences interspersed with MG-block sequences. Only the G units seem to be involved in intermolecular cross-linking during polymerization. Sodium alginate is widely used as a hydrogel.

Regarding alginate, and according to what is mentioned above, an alginate enriched in M units will be more flexible because the chain will have a more linear configuration, while a gel containing more G units will be more rigid because it will be more polymerized. In the context of the present invention, the alginate used has, for example, an M/G ratio between 1 and 2, in particular between 1 and 1.9 or between 1 and 1.5. In the context of the present invention, the alginate used has, for example, an M/G ratio of 1.9.

Preferably, the gelatin contained in the hydrogel is type A.

Gelatin is a collagen-derived macromolecule that contains bioactive sequences such as the RGD (arginine-glycine-aspartic acid) motif for cell adhesion. It is obtained by denaturation of the native triple helix structure of collagen via an acid (type A gelatin) or alkaline (type B gelatin) treatment. The amino acid composition of gelatin is similar but different from that of collagen following denaturation (deamination of glutamine to glutamic acid in the manufacturing process of type B gelatin). The structure of gelatin changes during gelation.

The preparation of hydrogels is well known in the art (E. M. Ahmed; Journal of Advanced Research, 2015, 6, 105-121), as well as the polymerization and cross-linking of alginate and gelatin (Chen Q, Tian X, Fan J, Tong H, Ao Q, Wang X. An Interpenetrating Alginate/Gelatin Network for Three-Dimensional (3D) Cell Cultures and Organ Bioprinting. Molecules. 2020; 25(3):756.)).

Preferably, the alginate is cross-linked with a cross-linking agent selected from divalent cations, in particular non-toxic cations. According to one embodiment, the divalent cation is selected from the group comprising or consisting of calcium, strontium, barium, zinc, copper, iron and nickel. According to one embodiment, the divalent cation is selected from the group comprising or consisting of calcium, strontium and barium. Preferably the divalent cation is calcium.

Preferably, the gelatin is cross-linked by any enzymatic method, physical such as UV light, or chemical, and in particular by an enzymatic method performed with an agent capable of forming covalent bonds between lysine and glutamine residues, and most preferably by a transglutaminase.

The enzyme transglutaminase (TAG) is an extracellular aminoacyltransferase. It is a monomeric protein with a single catalytic cysteine residue (active site). In the context of the invention, the gelatin of the hydrogel is preferably cross-linked with a type 2 transglutaminase. In particular, this TAG is commercially produced as a recombinant microbial protein by fermentation of the microorganism Streptoverticillium moboarense.

According to the present invention, the alginate and gelatin that are in the hydrogel that is comprised in the implant are cross-linked, i.e., transformed from a linear polymer into a three-dimensional polymer through the action of a cross-linking agent among those mentioned above.

In a particularly preferred embodiment, the implant according to the invention comprises a hydrogel comprising from 0.5% to 3% alginate and from 1% to 17.5% gelatin, and even more preferably from 1% to 2.5% alginate and from 2% to 10% gelatin. Advantageously, the hydrogel comprises 2% alginate and 5% gelatin.

Unless otherwise indicated, the percentages mentioned in the present description are expressed in mass/volume and relative to the total composition.

Preferably, in the hydrogel of the implants of the invention, the cross-linked alginate and gelatin are present in a weight ratio ranging from 1:0.3 to 1:35, and most particularly in a weight ratio of 1:2.5, respectively.

The hydrogel of the implants of the invention may also comprise, in addition to alginate and gelatin, fibrinogen which is also cross-linked.

The fibrinogen monomer is composed of two repeats of three α, β and γ chains connected by a central E domain and two fibrinopeptides A and B (FpA, FpB) connecting the a chains to the E domain It has a large number of cell adhesion motifs and thus allows increased cell development within the hydrogel.

In this case, the hydrogel will preferably comprise from 0.0001% to 6% cross-linked fibrinogen, and in particular 2% cross-linked fibrinogen.

According to a particularly preferred embodiment, the hydrogel of the implants of the invention is composed of cross-linked alginate and gelatin, or cross-linked alginate, gelatin and fibrinogen, without any other constituent capable of forming a gel.

Preferably, the hydrogel of the implants of the present invention contains cross-linked alginate, gelatin and fibrinogen in a weight ratio ranging from 1:0.3:0.00003 to 1:35:12, and most particularly in a weight ratio of 1:1:2.5, respectively.

The implants of the invention advantageously contain alginate, gelatin and optionally fibrinogen as natural constituents of the hydrogel. Nevertheless, other natural components may also be present in the implants of the invention, including in particular: chitin, chitosan, cellulose, agarose, chondroitin sulfate, hyaluronic acid, glycogen, starch, pullulan, carrageenan, heparin, collagen, albumin, fibrin, fibroin, dextran, xanthan, gellan, any component extracted from extracellular matrix such as collagens, laminin, proteoglycans such as Matrigel, GelMa type methacrylate gelatin.

In one embodiment, said natural components are present in concentrations ranging from 0.001% to 50%, preferably from 0.01% to 25%, or even more preferably from 0.1% to 10%.

In addition to the constituents of natural origin, and in particular those listed above, the implants of the present invention may also contain synthetic components, such as polyolefins (PE, PP, PTI-B, PVC), silicone (PDMS), polyacrylates (PMMA, pHEMA), polyester (PET, dacron, PGA, PLLA, PLA, PDLA, PDO, PCL), polyethers (PEEK, PES), polyamides, polyurethanes, PEG, pluronic F127.

In one embodiment, said synthetic components are present in concentrations ranging from 0.001% to 50%, preferably from 0.01% to 25%, or even more preferably from 0.1% to 10%.

Textile fibers of natural or synthetic origin may also be present in the implant composition.

Examples of fibers of natural origin include, but are not limited to, cellulose fibers.

Examples of fibers of synthetic origin include, but are not limited to, polyester fibers, nylon fibers, polyethylene fibers, polypropylene fibers, and acrylic fibers.

In one embodiment, said fibers are present at a concentration of less than 20%, preferably less than 10%, most preferably less than 5%. According to one embodiment, the implants of the invention do not comprise fibers, whether of natural or synthetic origin.

The implants according to the invention are acellular, i.e. they are free of any cell, and in particular of any living cell, during their manufacture. Nevertheless, the implants of the invention can be colonized by living cells after their manufacture, which makes it possible to avoid any manufacturing constraints related to the preservation of cell survival, proliferation and/or differentiation, and to carry out in vitro colonization of the implant once it has been manufactured but before it is implanted in the host organism in order to optimize its integration.

Examples of particular embodiments of implants according to the invention are thus implants comprising:
a hydrogel consisting solely of alginate and gelatin, without fibrinogen;
a hydrogel consisting of alginate, gelatin and fibrinogen;
a hydrogel consisting of alginate, gelatin and collagen;
a hydrogel consisting of alginate, gelatin, collagen and fibrinogen.

Particularly preferred embodiments within the scope of the invention relate to the implants defined in the following Table 3:

TABLE 3

| | | | | Young's | |
|---|---|---|---|---|---|
| | | | | modulus | |
| | | | | for the | |
| | | | | hydrogel | |
| | Alginate | Gelatin | Fibrinogen | (kPa) | Pore size |
| Implants A | 2% | 5% | 0% | 100 | 3 respective pore |
| | | | | | size zones: |
| | | | | | 5000-7000 μm; |
| | | | | | ~4000 μm; ~3000 μm, |
| | | | | | ou 1000-2500 μm; |
| | | | | | ~800 μm; ~250 μm |
| Implants B | 2% | 5% | 0% | 100 | 2 respective pore |
| | | | | | size zones: |
| | | | | | 500-7000 μm; |
| | | | | | ~3000 μm, ou |
| | | | | | 1,000-2500 μm; |
| | | | | | ~800 μm |
| Implants C | 2% | 5% | 0% | 100 | 1 pore size zone |
| | | | | | 2000-10000 μm |

In one embodiment, the implants of the invention are obtained by a manufacturing process in which alginate and gelatin are consolidated by cross-linking with at least one divalent cation, preferably calcium, and transglutaminase.

In one embodiment, said consolidation is done sequentially, i.e., the above-mentioned cross-linking agents are not added at the same time during the consolidation.

In one embodiment, the hydrogel once prepared is contacted with a solution comprising a divalent cation, preferably calcium, and then with a solution comprising transglutaminase. According to another embodiment, the hydrogel once prepared is contacted with a solution comprising transglutaminase and then with a solution comprising a divalent cation, preferably calcium.

In one embodiment, said consolidation is done concomitantly, i.e., the above-mentioned cross-linking agents are added at the same time during the consolidation.

In a particularly advantageous embodiment, the implants of the invention are obtained by a manufacturing process in which alginate and gelatin are consolidated by cross-linking with a solution comprising at least one divalent cation, preferably calcium, and transglutaminase.

Within the scope of the present invention, said solution may be obtained by alternative but nevertheless equivalent processes. The consolidation solution can be obtained by adding the different elements, i.e. at least one divalent cation, preferably calcium, and transglutaminase, in the same solution, or by mixing at least two solutions: a solution comprising at least one divalent cation, preferably calcium, and a solution comprising at least transglutaminase.

In one embodiment, during consolidation, the contacting of the hydrogel with the above-mentioned solution(s) is performed by immersion, during which the hydrogel is immersed in the entirety of the above-mentioned solution(s). It can also be done by soaking, spraying, drip, trickle, or similar.

For example, the hydrogel once prepared is contacted with a consolidation solution comprising at least one divalent cation, preferably calcium, and transglutaminase. During this consolidation, the contacting of the hydrogel with the consolidation solution can be performed by immersion, during which the hydrogel is immersed in its entirety in the consolidation solution. It can also be done by soaking, spraying, drip, trickle, or similar.

When the hydrogel contains fibrinogen in addition to alginate and gelatin, the consolidation further comprises cross-linking of the fibrinogen with thrombin. This cross-linking can be done sequentially with the cross-linking of alginate and gelatin (e.g., before or after the cross-linking of alginate and gelatin) or concomitantly.

In one embodiment, when the hydrogel contains fibrinogen in addition to alginate and gelatin, the hydrogel once prepared is contacted with a solution comprising a divalent cation, preferably calcium, then with a solution comprising transglutaminase, then with a solution comprising thrombin.

In one embodiment, when the hydrogel contains fibrinogen in addition to alginate and gelatin, the hydrogel once prepared is contacted with a solution comprising a divalent cation, preferably calcium, then with a solution comprising thrombin, then with a solution comprising transglutaminase.

In one embodiment, when the hydrogel contains fibrinogen in addition to alginate and gelatin, the hydrogel once prepared is contacted with a solution comprising transglutaminase, then with a solution comprising a divalent cation, preferably calcium, then with a solution comprising thrombin.

In one embodiment, when the hydrogel contains fibrinogen in addition to alginate and gelatin, the hydrogel once prepared is contacted with a solution comprising transglutaminase, then with a solution comprising thrombin, then with a solution comprising a divalent cation, preferably calcium.

In one embodiment, when the hydrogel contains fibrinogen in addition to alginate and gelatin, the hydrogel once prepared is contacted with a solution comprising thrombin, then with a solution comprising transglutaminase, then with a solution comprising a divalent cation, preferably calcium.

In one embodiment, when the hydrogel contains fibrinogen in addition to alginate and gelatin, the hydrogel once prepared is contacted with a solution comprising thrombin, then with a solution comprising a divalent cation, preferably calcium, then with a solution comprising transglutaminase.

In one embodiment, when the hydrogel contains fibrinogen in addition to alginate and gelatin, the consolidation solution comprises at least one divalent cation, preferably calcium, transglutaminase, and thrombin.

In a preferred embodiment, the implants of the invention are obtained by a manufacturing process in which the consolidation step, which consists of bringing the hydrogel into contact with the consolidation solution(s), is carried out at a temperature ranging from 15° C. to 40° C., and preferably from 20° C. to 40° C. and even more preferably from 21° C. to 37° C. According to another preferred embodiment, but which can also be combined with the above one regarding the temperature conditions, the implants of the invention are obtained by a manufacturing process during which the consolidation step, which consists of bringing the hydrogel into contact with the consolidation solution(s), is carried out for a period ranging from 10 minutes to 6 h, in particular from 30 minutes to 6 h, and ideally for 1 h to 3 h. Thus, according to an advantageous embodiment, the implants of the invention are obtained by a manufacturing process during which the consolidation step is carried out at 37° C. for 1 h30.

In one embodiment, the hydrogel is shaped prior to consolidation.

The implants of the present invention can be manufactured and shaped simultaneously, in particular by any volume structuring process (3D in particular), and in particular by adding or agglomerating material by stacking layers or successive deposition. Thus, according to one embodiment, the implant of the invention is obtained by an additive manufacturing process.

Among these processes, one may in particular mention the methods by injection, by extrusion, and in particular molding, 3D printing. Thus, according to one embodiment, the implant of the invention is obtained by extrusion of material, preferably 3D printing.

The implant may then be constituted by a plurality of layers each having a mesh made up of a plurality of meshes, the layers being stacked on top of one another in such a way that the meshes form the pores. According to one embodiment, said implant is formed of a number of layers between 2 and 3000.

In accordance with the aforementioned characteristics of the pores, the meshes of each layer may have homogeneous mesh sizes, i.e., not differing from each other by more than 15%.

In addition, the meshes of each layer may be homogeneously, i.e., evenly, distributed.

More particularly, the meshes of each layer may extend around central mesh axes having respectively homogeneous orientations, i.e. differing from each other by no more than 20°. The central mesh axes of the meshes of each layer may be arranged with homogeneous spacings, i.e., differing from each other by no more than 15%.

The meshes of each layer may respectively have homogeneous geometries, i.e., whose contours are superimposable with more than 50% of merged or parallel portions.

The meshes of each layer can be separated from each other by strands of material each having homogeneous thicknesses, i.e. not differing from each other by more than 15%.

In particular, the person skilled in the art will take care to choose a process which allows the shaping of materials with high viscosity, since a hydrogel consisting only of alginate and gelatin can have a viscosity ranging from 50 Pa·s to 6000 Pa·s, when measured at a temperature of 5° C. to 45° C.

In the context of the invention, the implants are preferably obtained by a 3D printing process. This technique also makes it possible to give the implant a suitable shape. Indeed, as previously indicated, the implants according to the invention have advantageous mechanical properties, and are particularly suited to their purpose.

By this technique, the implant can be “shaped” to match the physiognomy and/or wishes of the host. The implants of the invention thus provide “tailor-made” structured solutions whose dimensions and/or filling/porosity are defined with respect to the needs of the host body intended to receive the body implant and the role/function it will have to play in this recipient organism.

Thus, the present invention also relates to a three-dimensional body implant obtainable by a manufacturing process as described above. In particular, the body implant is obtainable by a manufacturing process comprising successively:
a step of preparing a hydrogel comprising gelatin and alginate,
a step of three-dimensional shaping of the hydrogel so as to form at least one porous zone, the porous zone comprising a plurality of pores each having a pore size, the porous zone having an overall porosity of between 100 μm and 10000 μm, the overall porosity corresponding to an average of the pore sizes measured in the porous zone, it being possible for the said shaping step to comprise, for example, the implementation of an additive manufacturing process, in particular of 3D printing, and
a step of cross-linking the hydrogel with at least one divalent cation, preferably calcium, and transglutaminase, said hydrogel having a mechanical strength of 1 kPa to 1000 kPa.

The process may further comprise a sterilization step.

According to particular provisions, the disclosure also relates to an implant that may have one or more of the following features:
a three-dimensional body implant which can have an overall porosity of at most 5000 μm and comprises a hydrogel comprising cross-linked alginate and cross-linked gelatin, the said hydrogel having a mechanical strength, also referred to here as elasticity or Young's modulus, of 1 kPa to 1000 kPa
a three-dimensional body implant that can have different porosity zones within its three-dimensional structure, in particular in the form of a gradient distributed over more than one zone of the implant
a three-dimensional body implant can also contain cross-linked fibrinogen,
a three-dimensional body implant which can be a breast implant which preferably has at least two, and in particular three, zones, each with different porosity,
a three-dimensional body implant which may comprise a hydrogel comprising cross-linked gelatin and cross-linked alginate, said hydrogel having a mechanical strength of 1 kPa to 1000 kPa and said implant having an overall porosity of at most 5000 μm,
wherein the gelatin can be cross-linked by an enzyme, preferably a transglutaminase,
a three-dimensional body implant that can have a porosity gradient distributed over more than one zone of the implant,
a three-dimensional body implant that can have a volume in the range of from 0.05 mL to 3 L, preferably from 100 mL to 600 mL,
a three-dimensional body implant may be a breast implant,
wherein the implant hydrogel may comprise from 0.5% to 3% alginate and from 1% to 17.5% gelatin,
wherein the implant hydrogel may further comprise cross-linked fibrinogen, preferably from 0.0001% to 6% fibrinogen,
a three-dimensional body implant obtainable by a process which comprises a step of cross-linking the hydrogel with a solution containing a divalent cation, preferably calcium, and an agent capable of forming covalent bonds between lysine and glutamine residues such as an enzyme, preferably a transglutaminase.
said solution may further comprise thrombin when the hydrogel comprises fibrinogen,
a three-dimensional body implant obtainable by a 3D printing process.

The invention also relates to a method of implementing the implant as described above in the context of reconstructive or cosmetic surgery, comprising a step of implanting the implant into the body of a subject.

Preferably, said implant is a breast implant. Said invention thus relates to a method of breast reconstruction comprising implanting in a subject in need thereof an implant according to the invention.

Said method may further comprise a step of injecting cells, preferably autologous cells, into said implant prior to its implantation in the subject.

According to one embodiment, the subject is a female. According to an embodiment, the subject is a woman who has undergone a mastectomy.

### BRIEF DESCRIPTION OF THE FIGURES

Further features, purposes and advantages of the invention will be apparent from the following description, which is purely illustrative and non-limiting, and which should be read in conjunction with the attached drawings in which:

FIG. 1 is a schematic representation of an implant according to the invention, of the breast type, which has a pore size gradient distributed over three zones.

FIG. 2 represents the comparison of the Young's modulus (A) and viscosity (B) of AG and FAG hydrogels that constitute the implants according to the invention.

FIG. 3 shows the comparison of the Young's modulus E0 (Pa) of an AG hydrogel in which the gelatin is cross-linked with and without transglutaminase and stored for up to 7 days at 37° C.

FIG. 4 represents the comparison of Young's modulus E0 (Pa) of an AG hydrogel and commercial hydrogels cross-linked or not with transglutaminase. *: Liquid compound at 37° C.; +: visible polymerization but insufficient gel stiffness at 37° C. for DMA measurement

FIG. 5 represents the viability and cell growth measured kinetically on FAG and AG hydrogels which constitute the implants according to the invention, and which were colonized in vitro by fibroblasts, after their manufacture.

FIG. 6 represents the viability and cell growth measured kinetically on FAG and AG hydrogels which constitute the implants according to the invention, and which have been colonized in vitro by adipose tissue stem cells, after their manufacture.

FIG. 7 represents the metabolic activity of AG implants according to the invention at different culture points following their in vitro colonization by a purified adipose tissue fraction, after their manufacture.

FIG. 8 represents the histological analyses by Hematoxylin, Phloxin, Saffron (HPS) staining of AG implants according to the invention after 2 days (4 images on the left) or 7 days (2 images on the right) of in vitro incubation with a fraction of purified adipose tissue, after their manufacture (Top: external edges of the matrices; Bottom: internal pores of the matrices; images taken in white light; magnification 100×; scale 100 μm).

FIG. 9 represents perilipin-1 immunostaining and Dapi staining of cell nuclei, on AG implants according to the invention after 2 days (top image) or 7 days (bottom image) of in vitro incubation with a purified adipose tissue fraction, after their manufacture (fluorescence imaging; magnification 200×; scale 50 μm).

FIG. 10 represents the comparison of Young's moduli of AG implants for varying durations of cross-linking at 21° C. (B) and 37° C. (A).

FIG. 11 represents the comparison of Young's moduli E0 and viscosities of AG and FAG implants after cross-linking with different concentrations of CaCl2 (A, D), TAG (B, E) and thrombin (C, F).

FIG. 12 represents the comparison of Young's modulus E0 (A-B) and viscosities (C-D) of AG and FAG implants after sequential or concomitant cross-linking with CaCl2, TAG and thrombin.

FIG. 13 represents the comparison of Young's modulus E0 (A) and viscosity (B) of AG and FAG implants after cross-linking with a solution containing calcium chloride or barium chloride.

FIG. 14A illustrates the study of the variation of dimensions (A1-A2) and pores (A3-A4) of AG and FAG implants according to the invention before and after cross-linking.

FIG. 14B illustrates the impact of sterilization on the dimensions (B1-B2) and Young's modulus (B3-B4) of these implants.

FIG. 15 illustrates the repeatability of the production of AG implants according to the invention in terms of dimensions (A), volume (B) and pore size (C).

FIG. 16 illustrates the repeatability of the shrinkage of AG implants according to the invention after consolidation.

FIG. 17 illustrates the repeatability of shrinkage of AG implants according to the invention as a function of the sterilization method.

FIG. 18A illustrates the repeatability of the extrusion diameter. FIG. 18B illustrates the repeatability of pore length (B1-B2) of AG implants according to the invention.

FIG. 19 represents images of varying pore sizes in an AG implant according to the invention.

FIG. 20 represents the surgical plan (left) of the in vivo subcutaneous implantation (right) of AG and FAG implants according to the invention.

FIG. 21 represents the histological analyses after staining with Masson's trichrome on sections of AG implants according to the invention, after subcutaneous implantation in vivo in rat back sites for 3 weeks (images at low, medium and high magnification).

FIG. 22 represents the average pore length of implants produced with different pore sizes.

FIG. 23 represents the average pore length of implants produced with an increasing pore size gradient from the base to the top.

FIG. 24 represents the apparent Young's modulus values of the different subparts of implants produced with different pore sizes.

FIG. 25 shows the compression tests on whole dentures with different architectures, stress-displacement curves.

FIG. 26 shows microscopic observation of the implant base without (left) or with (right) the addition of a perimeter.

FIG. 27A represents images of the 3D printing of a large-volume A/G implant (9 cm long, 7 cm wide, and 2.7 cm thick implant), the resulting implant after cross-linking, and the large pores obtained in the structure. FIG. 27B represents images of the 3D printing of a large volume A/G implant (12.6 cm diameter implant, and 5.3 cm thickness), the resulting implant after cross-linking and the large pores obtained in the structure.

FIG. 28 represents the macroscopic observation of the pores of implants with different filling rates.

FIG. 29 represents the average distance between the centers of the pores of implants with different filling rates.

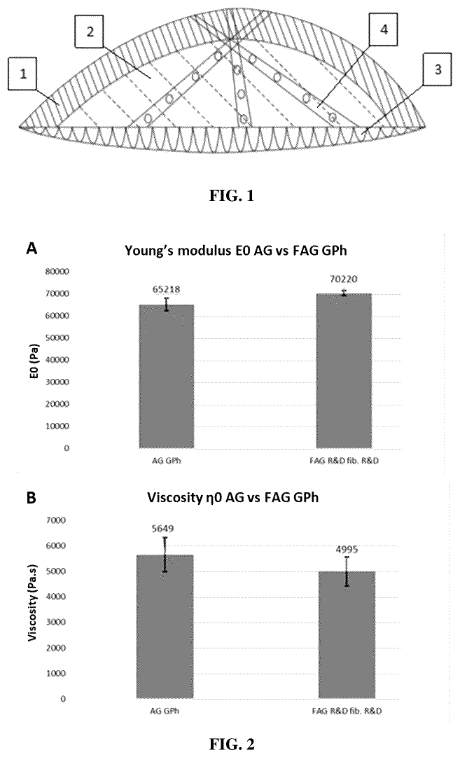

### EXAMPLES

The present invention will be better understood by reading the following examples which non-limitatively illustrate the invention.

### Materials and Methods

Protocol #1 Preparation of an AG hydrogel: In order to prepare the AG hydrogel, 2 g of alginate (very low viscosity, Alpha Aesar, France), 5 g of gelatin (Sigma-Aldrich, France) are dissolved at 37° C. for 12H in 100 mL of a 0.1M NaCl solution (Labelians, France).

Protocol #2 Preparation of a FAG hydrogel: In order to prepare the FAG hydrogel, 2 g of alginate (very low viscosity, Alpha Aesar, France), 5 g of gelatin (Sigma-Aldrich, France) and 2 g of fibrinogen (Sigma-Aldrich, France) are dissolved at 37° C. for 12 H in 100 mL of a 0.1M NaCl solution (Labelians, France).

Protocol #3 Moulding of an AG and FAG hydrogel: 1.8 mL of the hydrogel prepared according to protocol #1 and #2 are deposited in the wells of a 6-well culture plate and incubated at 21° C. for 30 minutes.

Protocol #4 Cross-linking of an AG hydrogel: A cross-linking solution is prepared by dissolving 4 g of Transglutaminase (Ajinomoto, Japan), 3 g of CaCl2 (Sigma Aldrich, France) in 100mL of a 0.1M NaCl solution (Labelians, France). The cross-linking solution is then put in contact with the hydrogel for 1 H30 at 37° C. (unless otherwise specified).

Protocol #5 Cross-linking of a FAG hydrogel: A cross-linking solution is prepared by dissolving 4 g of Transglutaminase (Ajinomoto, Japan), 3 g of CaCl2 (Sigma Aldrich, France) and 400 Units of thrombin (Sigma Aldrich, France) in 100 mL of a 0.1M NaCl solution. The cross-linking solution is then put in contact with the hydrogel for 1 H30 at 37° C. (unless otherwise specified).

Protocol #6 Dynamic Mechanical Analysis (DMA) in compression: The mechanical properties of FAG and AG hydrogels are measured in triplicate with a rotational rheometer (DHR2, TA Instrument, France), a Peltier plane (TA Instrument, France) and an 8 mm notched geometry (TA Instrument, France). Three 8 mm diameter disks are cut from the molded hydrogels according to protocol #3. The disk is placed on the lower geometry at 37° C. for 60 seconds and then a 10 μm oscillatory compression procedure is performed from 0.1 to 10 Hz at 100 μm/s and 37° C. The values of Young's modulus EO (Pa) and viscosity η0 (Pa·s) of the hydrogel are obtained from a visco-hyperelastic solid modeling using the E′ and E″ values acquired during the test.

Protocol #7 3D printing of hydrogels: Hydrogels prepared according to protocol #1, #2 are transferred into a 30 mL cartridge (Nordson EFD) equipped with a 410 μm diameter extrusion nozzle (Nordson EFD). The cartridge-nozzle assembly is then placed in a 3D printer (BioassemblyBot, Advanced Solution Lifescience, USA) allowing constant pressure to be applied to the cartridge while moving in all three directions of space. The printing parameters are a speed of 10 mm/sec, a pressure of 25-35 PSI and a temperature of 21° C. The different filling rates are obtained by the internal slicer of the printer control software (Tsim, Advanced Solution Lifescience, USA).

Protocol #8 In vivo implantation in rats: The in vivo implantation studies in rats were conducted on the BIOVIVO—Institut Claude Bourgelat (Lyon, France) preclinical research technical platform. The experiments were conducted in accordance with the European Directives 2010/63/EU. The 16 animals (Sprague Dawley rat, 250-300 g) were anesthetized by inhalation (oxygen and 5% isoflurane). The dorsal implantation sites were shaved and disinfected with povidone and sterile gauze, and sterile drapes were placed to delineate the surgical area. General anesthesia was maintained with isoflurane (2%) and oxygen inhalation. Pre-surgical analgesia was performed subcutaneously with meloxicam and morphine at 1 mg/kg respectively. Body temperature and pulse rate of the rats were monitored during surgery. Two skin incisions of 2-3 cm were made in the back region. A bioprosthesis was implanted in the dorsal subcutaneous region of each animal. The control group was performed with only the incision and dissection. In one animal per group, 4 surgical sites were performed, three bioprostheses and one control specimen. The surgical site was closed in layers using subcutaneous and cutaneous sutures with absorbable braided sutures (PDS® polidioxanone, 4/0 and Nylon 3/0, Ethicon J&J). Postoperatively, the animals were monitored for signs of suffering, and the surgical wounds were inspected daily for skin healing and absence of infection. Explantation took place 21 days after implantation.

Protocol #9 Histological analysis: Implants were fixed for 24 hours in a 4% formalin solution (Alphapat, France) and then dehydrated by successive baths of absolute ethanol (vwr chemicals, France) and methylcyclohexane (vwr chemicals, France) with an STP 120 dehydrator (Myr, Spain) and then embedded in kerosene (Sakura, Japan). Sections of 5 μm thickness were made with a HM 340e microtome (Microm, France). Hematoxylin Phloxine Saffron (HPS), Masson's Trichrome and DAPI staining were performed.

Protocol #10 Dynamic mechanical analysis (DMA) in compression: The mechanical properties of FAG and AG hydrogels were measured in triplicate with a rotational rheometer (DHR2, TA Instrument, France), a Peltier plane (TA Instrument, France) and a 25 mm geometry (TA Instrument, France). Punches of 25 mm diameter are cut in the implants produced according to the protocol #9. The punch is placed on the lower geometry at 37° C. for 60 seconds and then a 10 μm oscillatory compression procedure is performed from 0.1 to 10 Hz at 100 μm/s and 37° C. The values of Young's modulus EO (Pa) and viscosity η0 (Pa·s) of the hydrogel are obtained from a visco-hyperelastic solid modeling using the E′ and E″ values acquired during the test.

Protocol #11 Total mechanical analysis of the implants in compression: Placement of the implants on a Lloyd tensile/compression machine with a 1 kN load cell and compression plates, a test speed of 10 mm/min is used.

### Example 1—Mechanical Properties of Alginate/Gelatin (AG) and Fibrinogen/Alginate/Gelatin (FAG) Hydrogels

AG and FAG hydrogels were prepared from protocols #1 and #2, molded according to protocol #3, and then cross-linked using protocols #4 and #5 to study their DMA mechanical properties using protocol #6.

The results are shown in FIG. 2 (A-B). The measured Young's modulus and viscosity values are similar between the AG hydrogel and the FAG hydrogel following their cross-linking by the process of the invention. The Young's moduli under the specific conditions of this study are around 68000 Pa.

### Example 2—Impact of Cross-Linking with Transglutaminase on the Mechanical Properties of Alginate/Gelatin Hydrogel (AG).

Molded samples of AG were prepared from protocols #1 and #3 and cross-linked from a variant of protocol #4. In this variant, the cross-linking solution is composed of a 30 mg/mL calcium chloride solution only or a 30 mg/mL calcium chloride and 40 mg/mL transglutaminase solution. Four gels of each condition were cast and tested in DMA on the same day and after 1, 4 and 7 days of storage at 37° C., respectively, in order to mimic physiological conditions.

The samples were then studied by DMA using protocol #6.

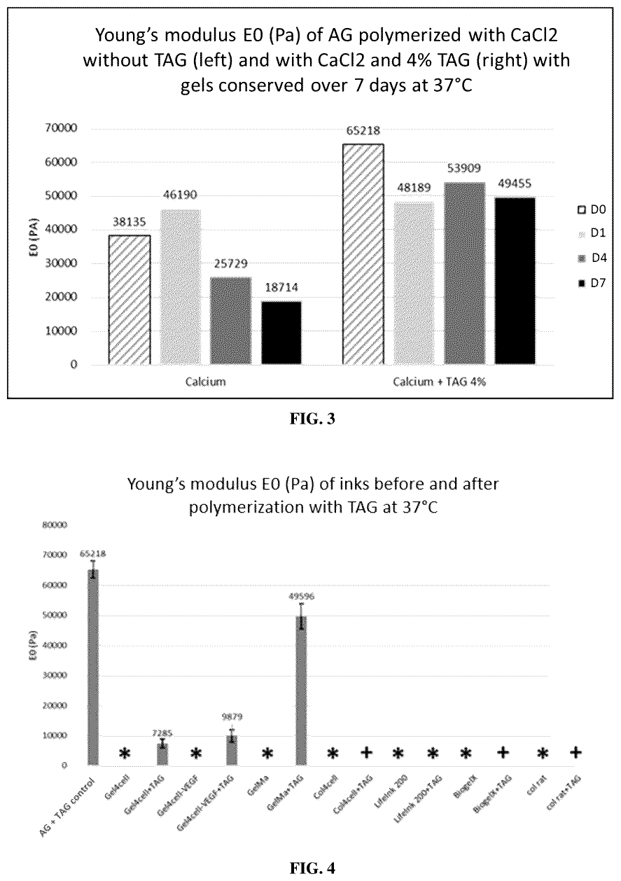

The results are shown in FIG. 3. This study shows the advantageous effect of the use of transglutaminase during cross-linking on the mechanical properties of hydrogels. This effect is even greater when the gels are converted at 37° C., justifying the particular interest of cross-linking according to the invention for hydrogels intended to be implanted.

### Example 3—Impact of Cross-Linking with Transglutaminase on the Mechanical Properties of Commercial Gelatin and/or Collagen Hydrogels

Molded samples of GA were prepared from protocols #1 and #3 and cross-linked from protocol #4. The commercial hydrogel samples listed in Table 4 below were prepared according to the protocols provided by the suppliers and molded according to protocol #3.

TABLE 4

| Hydrogel | | |
|---|---|---|
| commercial name | Reference | Supplier |
| Gel4Cell | BIS-101 | Bioink Solution |
| Gel4Cell-VEGF | BIS-103 | Bioink Solution |
| Col4Cell | BIS-108 | Bioink Solution |
| GelMA | VL5000000010 | Cellink |
| Lifeink 200 Type I Collagen | 5278-5ML | CellSystems |
| BiogelX | B5X-0000 | Biogelx |
| Rat tail Collagen | A10483-01 | Sigma |

Hydrogels were cross-linked with a variation of protocol #4, using either a solution comprising only calcium at 30 mg/mL (no TAG), or a solution of calcium at 30 mg/mL and transglutaminase at 40 mg/mL, in order to observe the impact of TAG.

The uncross-linked and cross-linked samples with TAG were then studied by DMA using protocol #6.

The results are grouped in FIG. 4. Six of the seven commercial hydrogels studied were cross-linked with transglutaminase. Collagen-based hydrogels (Col4Cell, Rat Collagen) are not stiff enough to be analyzed by DMA but gelatin-based hydrogels (Gel4cell, Gel4cell-VEGF and GelMa) have a significantly higher Young's modulus after transglutaminase cross-linking (7.3, 9.9 and 50 kPa respectively). This study shows the effect of cross-linking with transglutaminase on the stiffness of commercial hydrogels.

### Example 4—Influence of the Amount of Alginate and Gelatin in a Fibrinogen/Alginate/Gelatin (FAG) Hydrogel on Mechanical Properties

FAG hydrogels were prepared from a variant of protocol #2, molded according to protocol #3, then cross-linked thanks to protocol #5, then their mechanical properties were studied by DMA thanks to protocol #6. In this variant, we studied these mechanical properties by preparing the FAG hydrogel, with 1 or 3 or 2 g of alginate, and 10 or 7.5 or 5 g of gelatin, respectively, and 2 g of fibrinogen.

The results are grouped in Table 5 below. The Young's moduli under the specific conditions of this study range from 200 to 800 kPa.

TABLE 5

| | | | Young's modulus |
|---|---|---|---|
| Gelatin (%) | Alginate (%) | Fibrinogen (%) | (kPa) |
| 10 | 1 | 2 | 800 |
| 7.5 | 3 | 2 | 600 |
| 5 | 2 | 2 | 200 |
| 2 | 2 | 0 | 70 |
| 1 | 1 | 0 | 35 |

### Example 5—Evaluation of Fibrinogen/Alginate/Gelatin (FAG) and Alginate/Gelatin (AG) Hydrogels Colonization by Fibroblasts

AG and FAG hydrogels were prepared from protocols #1 and #2. Square implants of 1.5 cm side and 0.2 cm thickness were then printed using protocol #7 and cross-linked using protocols #4 or #5. The printed implants were produced with a 50% fill rate and an extrusion nozzle of 410 μm internal diameter. A negative control (empty well) is also used.

Normal human fibroblasts in passage 6 are thawed and amplified in 175 cm2 culture flasks in culture medium containing DMEM supplemented with 10% calf serum and 1% antibiotics. Each implant was seeded on its surface with a cell suspension of normal human fibroblasts at a concentration of 4000000 fibroblasts/ml. 250 μl of this suspension was drip-fed onto each implant, i.e. 1000000 fibroblasts/implant. After 1 hour of adhesion, the implants were immersed with culture medium. Implants were cultured in culture medium composed of DMEM containing 10% calf serum supplemented with vitamin C and EGF (Epidermal Growth Factor) at 37° C., 5% CO2. Implants were cultured with this same medium for 21 days, renewed every 3 days.

The metabolic activity of the fibroblasts within the implants was studied by colorimetric analysis with Alamar Blue on days 3, 5, 8, 10, 14 and 21 after seeding. The solution was made by diluting 10-fold a solution of Alamar Blue (DAL 1100, Invitrogen) in DMEM. After 19 hours of incubation at 37° C., 100 μl of the supernatants were collected and their absorbance at 570 nm and 600 nm was measured by spectrophotometer (NanoQuant® infinite M200PRO, TECAN).

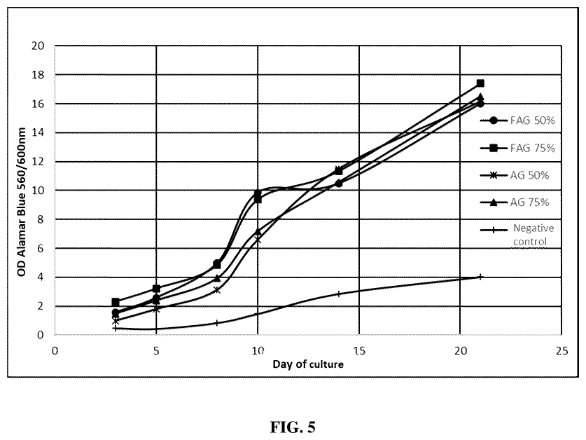

Cell viability and growth were monitored over 21 days of culture using 6-point kinetics on days 3, 5, 8, 10, 14 and 21. The results are shown in FIG. 5.

The results confirmed that all implants allowed fibroblast adhesion and survival as early as day 3 of culture. Cell growth is observable for each porous implant over the 21 days of culture, on both types of hydrogels (FAG and AG) as well as for each overall porosity employed.

### Example 6: Evaluation of Colonization of Fibrinogen/Alginate/Gelatin (FAG) and Alginate/Gelatin (AG) Hydrogels by Adipose Tissue Stem Cells (ASC)

AG and FAG hydrogels were prepared from protocols #1 and #2. Square implants of 1.5 cm side and 0.2 cm thickness were then printed using protocol #7 and cross-linked using protocols #4 or #5. The printed implants were produced with a 50% and 75% fill rate and an extrusion nozzle of 410 μm internal diameter. Sterilization was performed by the company IONISOS (France) by irradiating the implants with a dose of 30 kGy of Gamma ray.

Normal human adipocyte stem cells in passage 2 to 5 were thawed and amplified in 175 cm2 culture flasks in culture medium containing DMEM supplemented with 10% serum and 1% antibiotics. Each implant was seeded on its surface with a cell suspension of ASC at a concentration of 6, 12, or 24 million ASC/ml. 250 μl of these suspensions were drip-fed onto each implant, i.e. 1.5, 3 or 6 million ASC/implant. After 1 hour of adhesion, the implants were immersed with culture medium. Implants were cultured in culture medium containing DMEM supplemented with 10% serum and 1% antibiotics for 7 days and then in medium containing DMEM supplemented with 10% serum, insulin, rosiglitasone and 1% antibiotics for 14 days. The culture media are renewed every 3 days.

The metabolic activity of fibroblasts within the implants was studied by colorimetric analysis with Alamar Blue on culture days 3, 5, 7, 14 and 21 after seeding. The solution was made by diluting 10-fold a solution of Alamar Blue (DAL 1100, Invitrogen) in DMEM. After 5 hours of incubation at 37° C., 100 μl of the supernatants were collected and their absorbance at 570 nm and 600 nm was measured by spectrophotometer (NanoQuant® infinite M200PRO, TECAN).

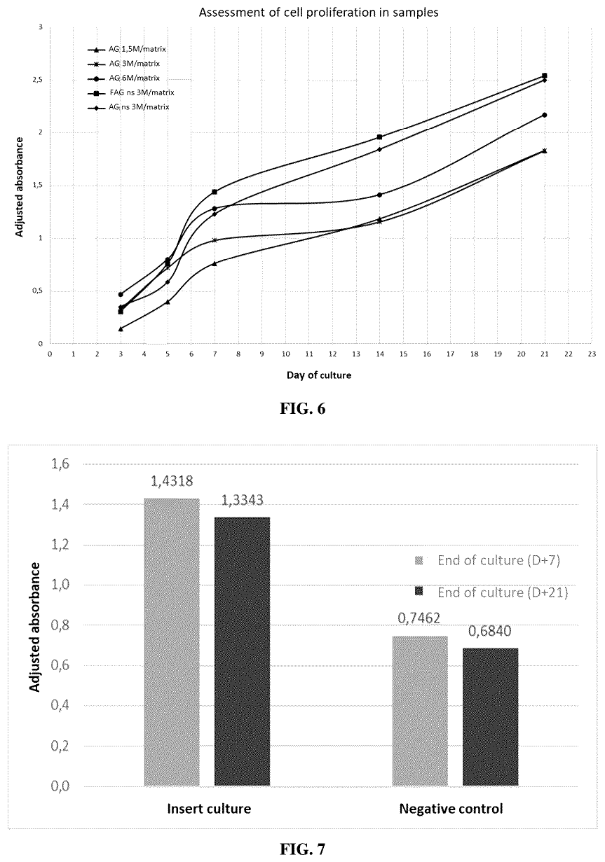

Cell viability and growth were monitored over 21 days of culture using 6-point kinetics on days 3, 5, 7, 14 and 21. The results are shown in FIG. 6.

The results confirmed that all implants allowed adipocyte stem cells to adhere and survive from day 3 of culture. Cell growth is observable for each porous implant over the 21 days of culture, on both types of hydrogels (FAG and AG) as well as for each density of seeding.

### Example 7: Evaluation of the Colonization of Alginate/Gelatin (GA) Hydrogels in Contact with a Purified Adipose Tissue Fraction

GA hydrogels were prepared from protocol #1. Cubic implants of 1.5 cm side and 0.8 cm thickness were then printed using protocol #7 and cross-linked using protocol #4. The printed implants were produced with a 50% fill rate and an extrusion nozzle of 410 μm internal diameter.

The lipoaspirate is centrifuged at 1500 RPM for 2 minutes and then rinsed with 1×PBS. The lipoaspirate was again centrifuged at 1500 RPM for 30 seconds and then the 1×PBS was removed. The lipoaspirate was considered purified.

Each implant was then immersed in 6 mL of purified lipoaspirate, and the whole set was placed in a culture insert in a 6-well plate with incubation in medium containing DMEM supplemented with 10% serum and 1% antibiotics at 37° C. 5% CO2 for 2 days or 7 days.

Following contact with lipoaspirate, the implants were grown in 6-well plates in culture medium containing DMEM supplemented with 10% serum, insulin, rosiglitasone, and 1% antibiotics, with 3 medium changes per week until 21 days.

Cellular metabolic activity within the implants was studied by colorimetric analysis with Alamar Blue on culture days 2, 7, and 21 after seeding. The solution was made by diluting 10-fold a solution of Alamar Blue (DAL 1100, Invitrogen) in DMEM. After 5 hours of incubation at 37° C., 100 μl of the supernatants were collected and their absorbance at 570 nm and 600 nm was measured with a spectrophotometer (NanoQuant® infinite M200PRO, TECAN).

Cell viability and growth were monitored over 21 days. The results are shown in FIG. 7. A much higher metabolic activity than the negative control was observed in the implants that were in contact with purified lipoaspirate.

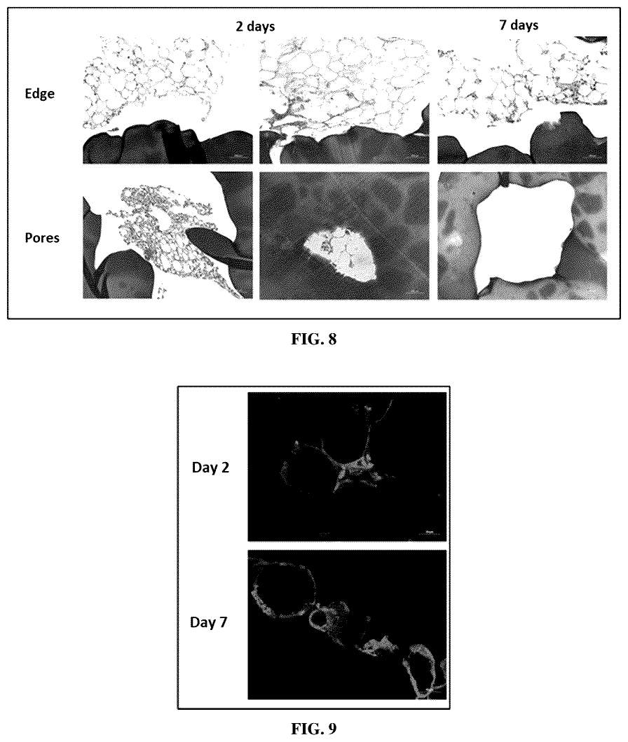

Histological analyses were performed to complete this study according to protocol #9. The results are shown in FIG. 8. The images reveal the presence of agglomerated, polygonal, uniform, unilocular, and bulky adipocytes. These morphological characteristics are those of healthy adipocytes, which can be found in adipose tissue.

Immunostaining for perilipin-1 was also performed. Samples were included in OCT (CellPath, KMA-0100-00A) and then stored at −80° C. Sections of 16 μm thickness were made for each sample with a cryostat (Microm, HM 520). The sections were then fixed in acetone/methanol (v/v) solution for 20 minutes and rinsed 3 times in 1×PBS. A 1-hour incubation at room temperature in 4% PBS-BSA solution was performed to saturate the aspecific sites. The sections were then incubated overnight at room temperature with perilipin-1-specific primary antibody solution. The next day, the sections were rinsed three times with 1×PBS and then incubated 45 minutes with Alexa fluor 568-coupled secondary antibody solution at room temperature. Then the sections were rinsed three times with 1×PBS and mounted between slide and coverslip with Dapi fluoromount-G® mounting medium (SouthernBiotech). The images obtained are grouped together FIG. 9.

The images show adipocytes with large spherical or polygonal vacuoles depending on the clustering of the cells. The adipocytes appear as unilocular and their size is also physiological as it ranges from 50 to 200 μm.

Taken together, these results confirm the adhesion, survival, and regeneration of a human adipose tissue brought into contact with the implants. The particular structure and composition of the implants thus form a favorable environment for the regeneration of healthy adipose tissue.

### Example 8—Influence of Temperature and Cross-Linking Time on the Mechanical Properties of an Alginate/Gelatin Hydrogel (AG)

Molded samples of AG were prepared from protocols #1 and protocol #3 and cross-linked from a variant of protocol #4. In this variation, the cross-linking times and temperatures were changed from 10 minutes to 14 H and from 37° C. to 21° C.

The samples were then studied by DMA using protocol #6.

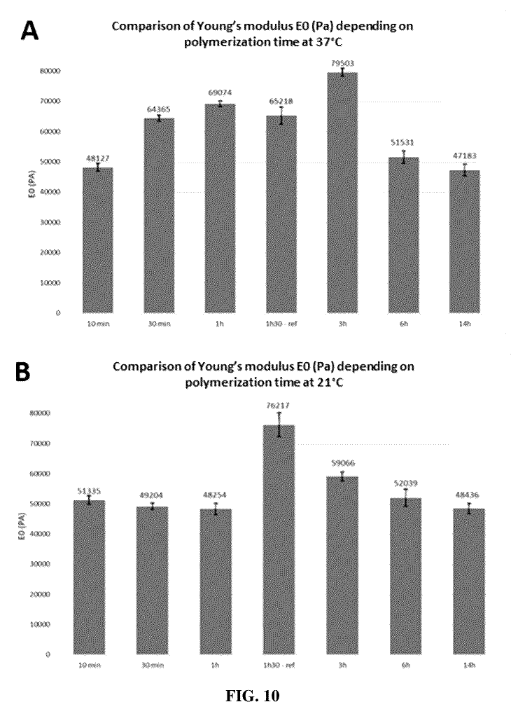

The results are shown in FIG. 10 (A-B). The cross-linking times as well as the cross-linking temperature have very little influence on the final mechanics (Young's modulus) of the hydrogels. However, it seems that an optimum can be found around 1:30, whatever the temperature.

These Young's moduli are also very stable over 7 days after cross-linking at 37° C. The cross-linking of the gelatin was efficient since there was no loss of dissolved gelatin in the medium.

### Example 9—Impact of Cross-Linking Solution Component Concentration on the Mechanical Properties of Alginate/Gelatin (AG) and Fibrinogen/Alginate/Gelatin (FAG) Hydrogels Once Cross-Linked

Molded samples of AG and FAG were prepared from protocols #1, #2 and #3, cross-linked from a variant of protocols #4 and #5. In this variation, the concentrations of the components of the cross-linking solution were changed (transglutaminase, calcium chloride, and thrombin).

The samples were then studied by DMA using protocol #6.

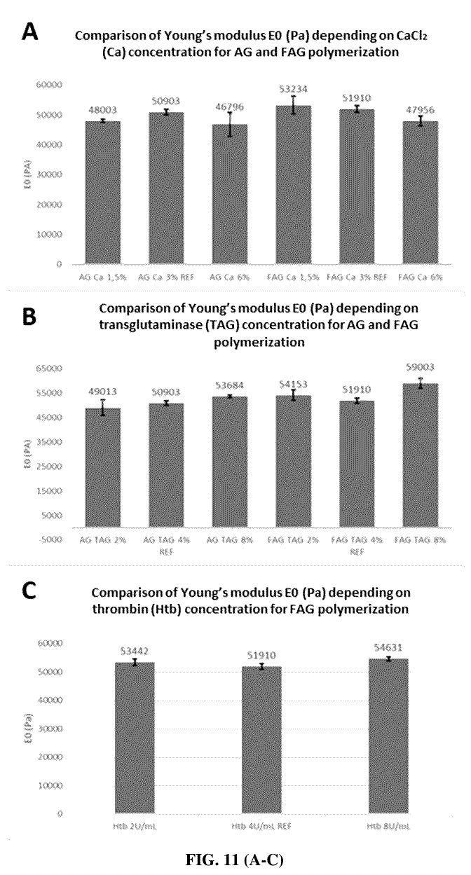

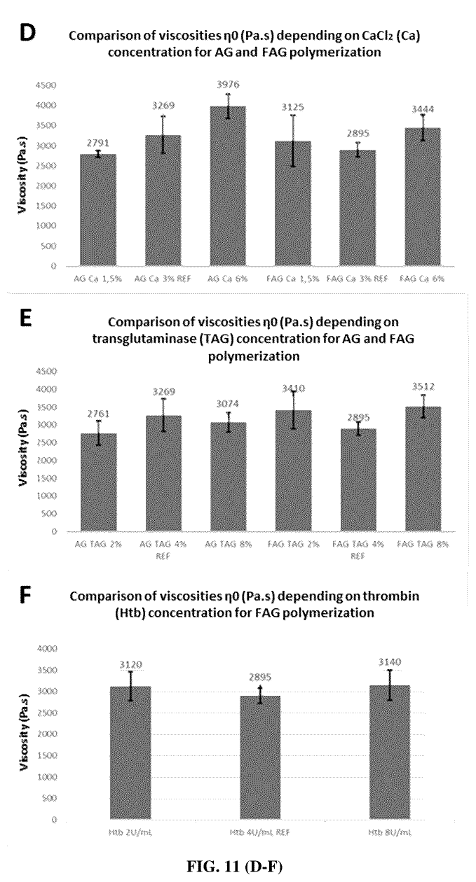

The results are grouped in FIG. 11 (A-F). Over this range of reagent concentrations, no significant variations were observed (E9 all very similar).

### Example 10—Impact of Sequential or Concurrent Cross-Linking of Alginate/Gelatin (AG) and Fibrinogen/Alginate/Gelatin (FAG) Hydrogel

Molded samples of AG and FAG were prepared from protocols #1, #2, and #3, cross-linked from a variant of protocols #4 and #5. In this variant, we investigated sequential cross-linking on FAG and AG, which involves cross-linking the hydrogel in several steps.

Each step took 1 h and three rinses with 0.1M NaCl solution were performed between each step to remove residual cross-linking agents.

The samples were then studied by DMA using protocol #6.

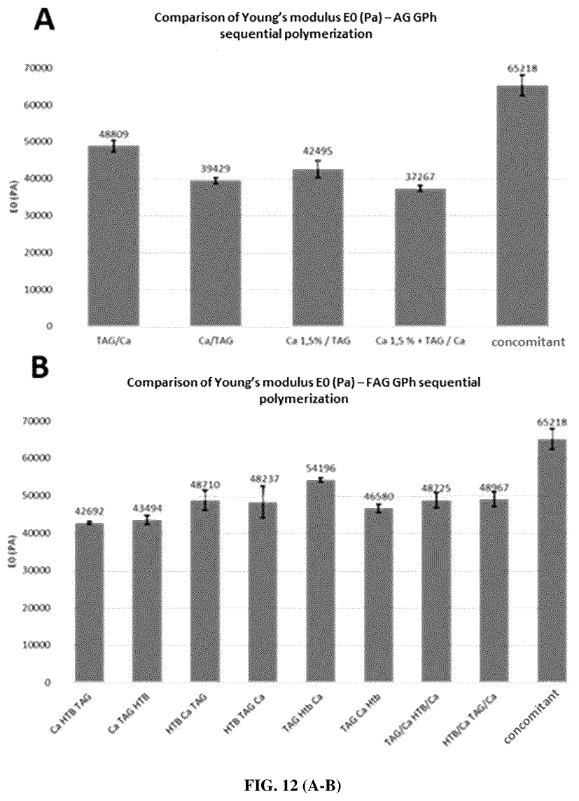

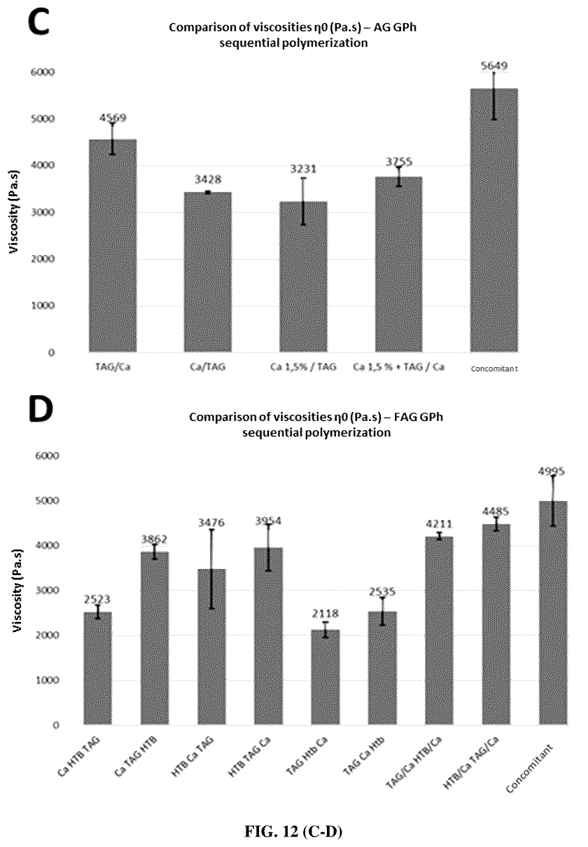

The results are shown in FIG. 12 (A-D). The sequential cross-linking set (FAG and AG) generate hydrogels with lower Young's moduli than the single step cross-linking.

It can be observed that very soft and brittle gels are obtained if calcium is not added first, indeed TAG and thrombin are calcium-dependent their activity is therefore largely decreased without the addition of CaCl2. The gels are therefore difficult to manipulate without the calcium cross-linking. When thrombin is added first, the gels have very little mechanical strength and holes appear.

### Example 11—Impact of the Nature of the Divalent Cation for the Cross-Linking of Alginate/Gelatin (AG) and Fibrinogen/Alginate/Gelatin (FAG) Hydrogels

Molded samples of AG and FAG were prepared from protocols #1, #2, and #3, cross-linked from a variant of protocols #4 and #5. In this variant, we studied a cross-linking in the presence of barium chloride 30 mg/mL.

The samples were then studied by DMA using protocol #6.

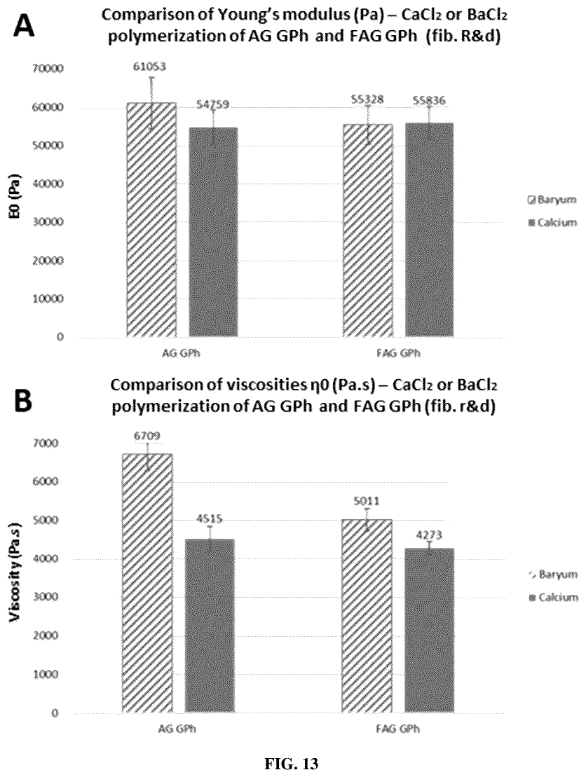

The results are shown in FIG. 13 (A-B). Cross-linking in the presence of barium results in gels with Young's moduli very similar to those obtained with CaCl2. However, since barium increases the viscosity of the gels, the formation of additional pendant chains can be assumed.

### Example 12—Maintenance of the Three-Dimensional Structure and Mechanical Properties of Alginate/Gelatin (AG) and Fibrinogen/Alginate/Gelatin (FAG) Hydrogel Implants After Sterilization

The AG and FAG hydrogels were prepared from protocols #1, #2 and #3, cross-linked using protocols #4 and #5, optically observed and then studied by DMA using protocol #6. The printed shapes are half-spheres of 2 cm diameter produced with variable filling rates (30, 50 and 75%).

Sterilization was performed by the company IONISOS (France) by irradiation of the implants with a variable dose (30 kGy and 40 kGy) of Gamma ray.

The impact of the cross-linking step on the dimensions of alginate/gelatin and fibrinogen/alginate/gelatin hydrogel implants was studied. These dimensions were measured from macroscopic images.

The dimensions of the pores obtained as a function of the filling rate were also studied. These dimensions were measured from images made with a microscope (Olympus, ×4 magnification).

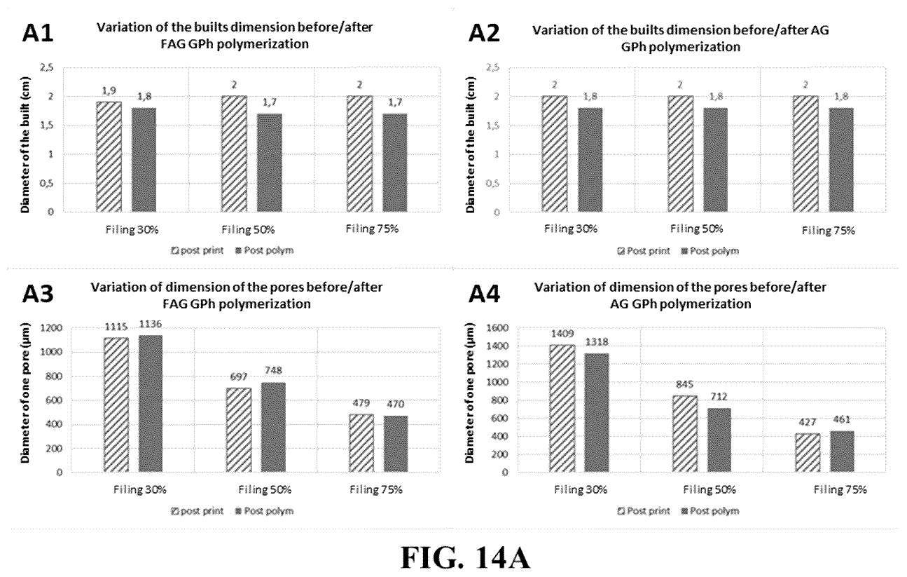

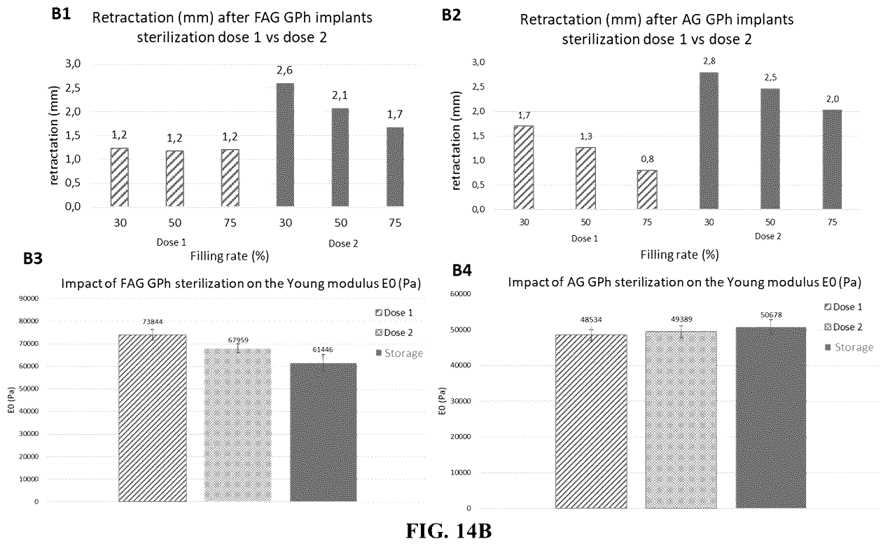

The results are shown in FIG. 14 (A-B). The implants shrink by an average of 10% following the cross-linking step. However, the pore size does not vary significantly (FIG. 14A (A1-A4)).

Regarding sterilization, it appears that the 40 kGy dose leads to a higher shrinkage of the constructs than the 30 kGy dose. Concerning EO, sterilization does not lead to any change in the mechanics of the material for both doses (FIG. 14B (B1-B4)).

### Example 13: Production Quality of Large Alginate/Gelatin (AG) Hydrogel Implant: Repeatability of Impression Dimensions, Dimensions After Consolidation and Sterilization of the Implant by Several Methods

AG hydrogels were prepared from protocol #1. Half-sphere shaped implants of 6 cm diameter and 2 cm thickness were then printed according to protocol #7 and cross-linked using protocol #4, then optically observed and measured. The printed shapes were produced with variable filling rates (25 to 65%) and extrusion nozzles of 410 or 840 μm internal diameter. Sterilization was performed by IONISOS (France) by irradiating the implants with 2 doses (30 kGy and 40 kGy) of Beta rays or a range dose of 30 kGy.

The impact of the cross-linking and sterilization step on the dimensions of large alginate/gelatin hydrogel implants was studied. These dimensions were measured from macroscopic images.

The dimensions of the pores obtained as a function of the filling rate were also studied. These dimensions were measured from images made with a microscope (Olympus, ×4 magnification).

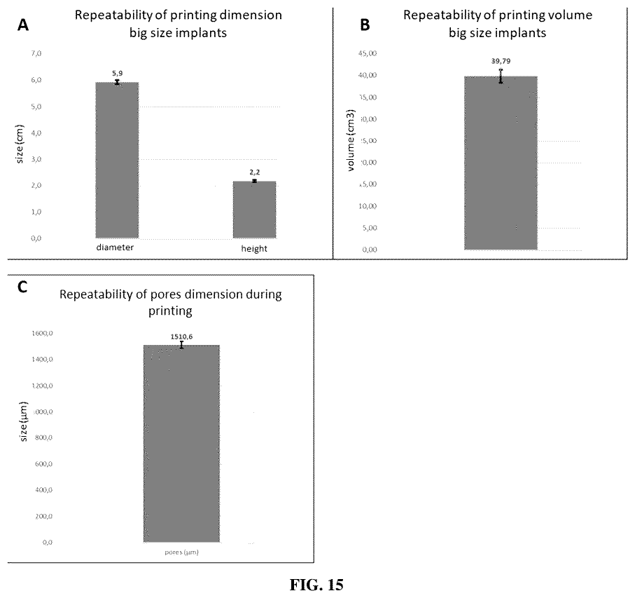

The results after printing are shown in FIG. 15 (A-C). These results show a high repeatability of the dimensions of the large 3D printed implants, reflecting a high production quality.

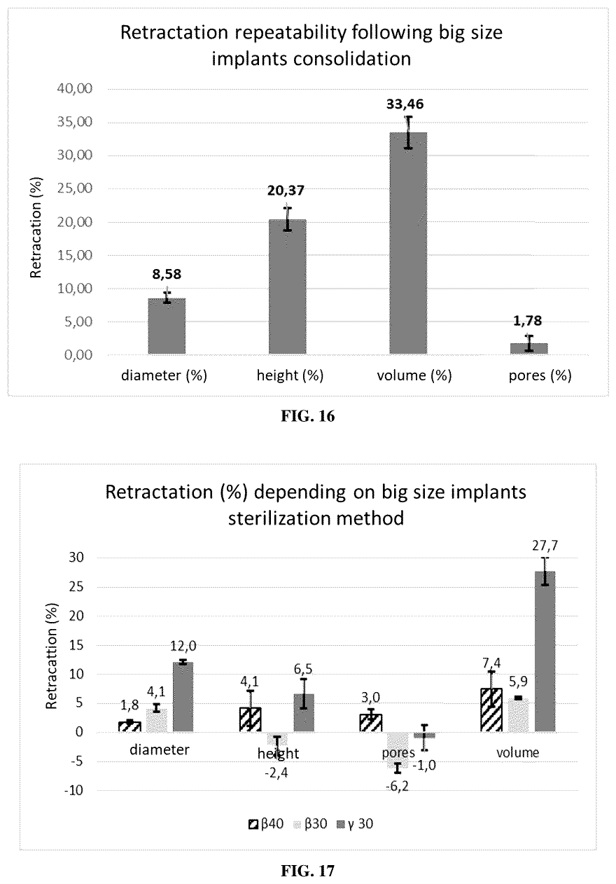

The results after consolidation of the implants are grouped FIG. 16. This graph shows a high repeatability of the shrinkage of the large implants after the consolidation stage.

The results after sterilization of the implants by 3 methods (β-rays doses 40 and 30 kGy and γ-rays 30 kGy) are grouped FIG. 17. These results show less shrinkage of the large implants with the β30 and 40 kGy rays.

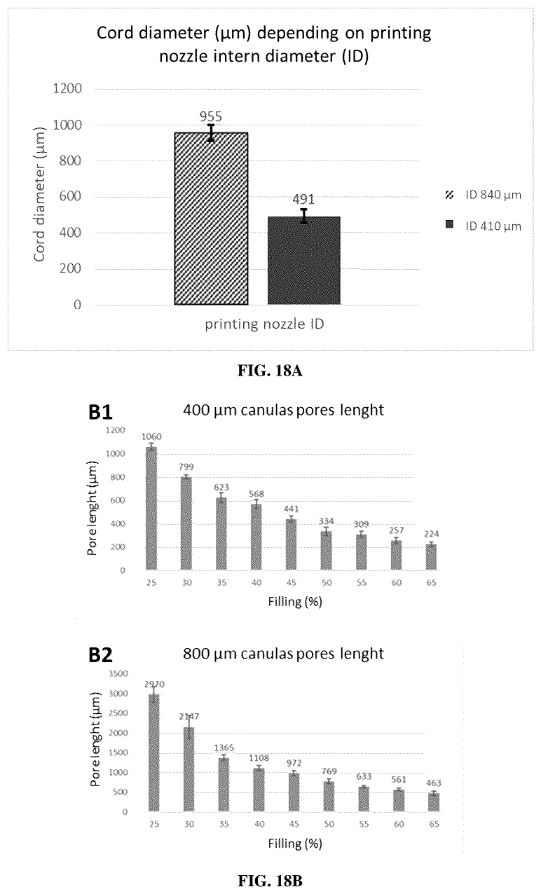

Large implants were printed with 2 extrusion nozzles with internal diameters of 410 and 840 μm with fill rates of 25 to 65%. The repeatability of the extrusion diameter as well as the obtained pore length were measured. The results are shown in FIG. 18 (A-B).

FIG. 18A shows the high repeatability of the extruded bead size. FIG. 18B (B1-B2) shows the variation of pore length with the filling rate of the hydrogel.

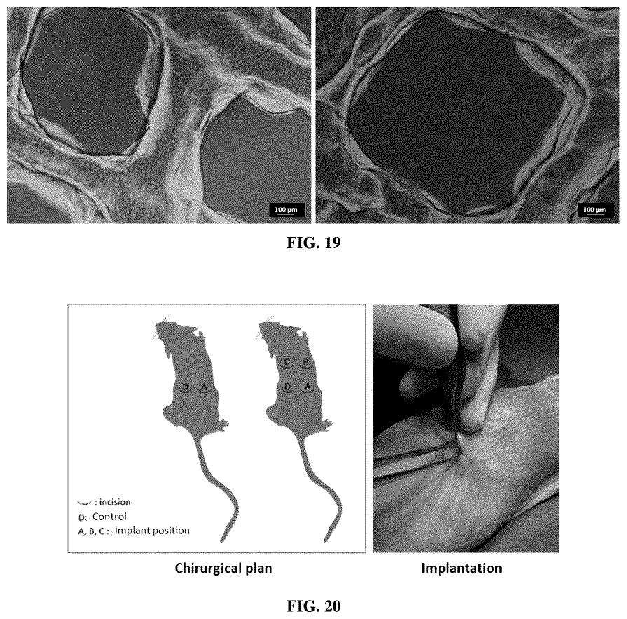

Images of varying pore sizes were taken and are grouped in FIG. 19.

These data show the wide range of pores that can be obtained for the implants and their high repeatability and production quality.

### Example 14—Studies of the Resistance of Implants In Vivo

GA and FAG hydrogels were prepared from protocols #1, #2 and #7, cross-linked through protocols #4 and #5. The printed shapes were 1 cm diameter half-spheres, produced with varying fill rates (30, 50 and 75%).

The porous half-spheres were sterilized with a dose of 30 kGy and then implanted subcutaneously in rats according to protocol #8.

Details of the implantation groups are described in the following Table 6, which refers to the surgical implantation plan described in FIG. 20.

TABLE 6

| Group | | | position of |
|---|---|---|---|
| (hydrogel/filling rate) | no animal | no implant | the implant |
| FAG 30% | 1 | 1 | A |
| | 2 | 2 | A |
| | 3 | 3 | A |
| | 4 | 4 | A |
| | | 5 | B |
| | | 6 | C |
| AG 30% | 5 | 7 | A |
| | 6 | 8 | A |
| | 7 | 9 | A |
| | 8 | 10 | A |
| | | 11 | B |
| | | 12 | C |
| AG 50% | 9 | 9 | A |
| | 10 | 14 | A |
| | 11 | 15 | A |
| | 12 | 16 | A |
| | | 17 | B |
| | | 18 | C |
| AG 75% | 13 | 19 | A |
| | 14 | 20 | A |
| | 15 | 21 | A |
| | 16 | 22 | A |
| | | 23 | B |
| | | 24 | C |

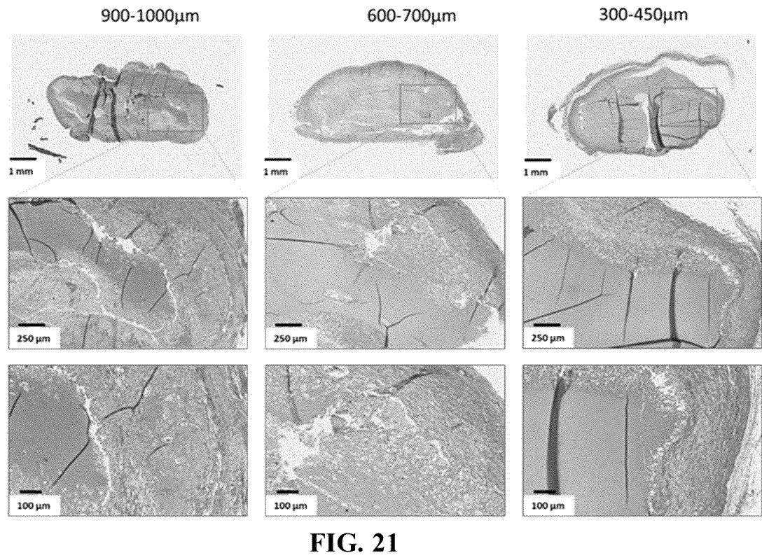

Histological analyses were performed using protocol #9, and the results are grouped in FIG. 21. Explantation was used to validate the resistance of the implants to skin tension. Histological analyses were used to assess cell colonization, vascularization, extracellular matrix synthesis, and the presence of areas of inflammation.

### Example 15—Production Quality of Large Implants with Various Pore Sizes and Study of the Impact of these Porosities on the Mechanical Properties of said Implants

Semi-anatomical breast prosthesis type implants (height: 8.83 cm; width: 6.37 cm; height: 2.86 cm) are produced using protocol #1 and a variant of protocol #7 (using a nozzle with an internal diameter of 840 μm) and then cross-linked using protocol #4. These implants are produced with different internal porosities:
Implants with one pore size for their entire volume.
Implants with two pore sizes distributed according to the fact that a first part at the base of the implant has one pore size and a second part at the top of the implant has another pore size.
Implants with three pore sizes distributed according to whether a first portion at the base of the implant (named base) has one pore size, another second portion (named core) in the middle and above the base of the implant has another pore size, and another third portion (named shell) on the surface and above the base of the implant has another pore size.
Implants with a gradient of pore sizes increasing from their bases have also been produced.

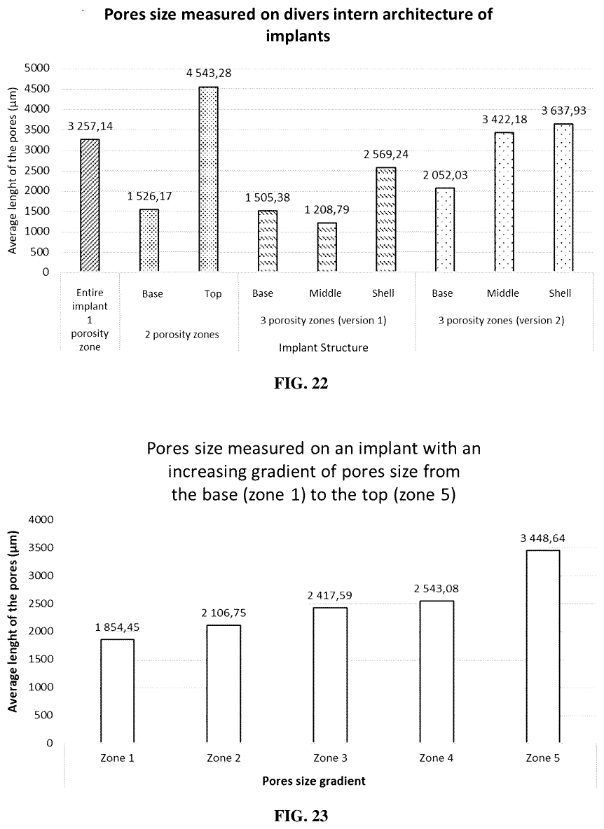

The dimensions of the obtained pores were studied. These dimensions were measured from images taken with a microscope (Olympus, ×4 magnification). The results of these measurements are shown in FIGS. 22 and 23. Pores of different sizes and very reproducible can be obtained in the different parts of the implant. A gradient of reproducible and increasing pore sizes from the base to the top can also be obtained.

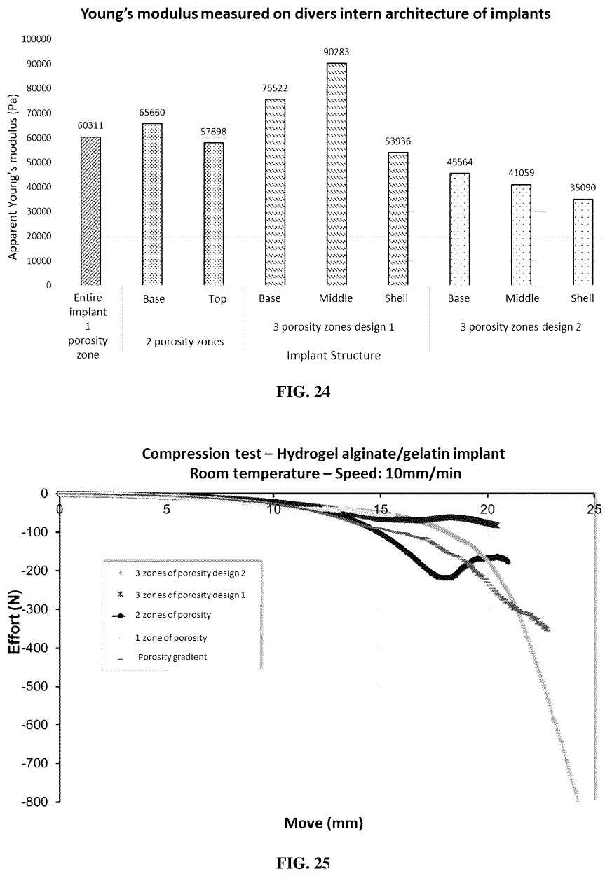

The mechanical properties of the subparts of these implants were studied by DMA according to protocol #11 and the mechanical properties of the implants were studied by total mechanical analysis according to protocol #12. The results of these measurements are shown in FIGS. 24 and 25. The Young's moduli observed vary inversely with respect to the pore size. Thus, for example, the decrease in the size of the pores at the heart of the implants allows a higher modulus to be obtained, reflecting a greater mechanical resistance. Thus, variations in pore size and the distribution of pore size zones make it possible to obtain a wide range of Young's modulus and therefore to obtain implants with greater or lesser strength. Concerning compression tests on whole prostheses, we observe that each configuration of pores brings different mechanical properties to the implants. Indeed, for an effort of −35N, the prosthesis with only 1 zone of porosity deformed less contrary to the prostheses with 3 zones of porosity which deformed more. The curves also allow us to identify different breaking behaviors. The prosthesis with only one porosity zone deformed progressively before breaking, whereas for the prosthesis with two porosity zones, the breaking was progressive and then brutal at −217 N, resulting in a stress recovery. Thus, by varying the pore sizes and the distribution of pore size zones, implants with different mechanical properties are obtained. This makes it possible to adapt the mechanical properties of the implants according to the desired application.

### Example 16—Addition of a Perimeter Around the Base of the Implant

Semi-anatomical sized breast prosthesis implants (Height: 8.83 cm; Width: 6.37 cm; Height: 2.86 cm) are produced from protocol #1 and a variation of protocol #7 (using an 840 μm inner diameter nozzle and adding a perimeter to the base of some implants) and then cross-linked from protocol #4. These implants all have a single pore size and a perimeter is added to some implants. This perimeter is characterized by the addition of a continuous filament surrounding the implant, tangentially to all the filaments located at the periphery of the implant. This perimeter is added on the first three layers of the implant.

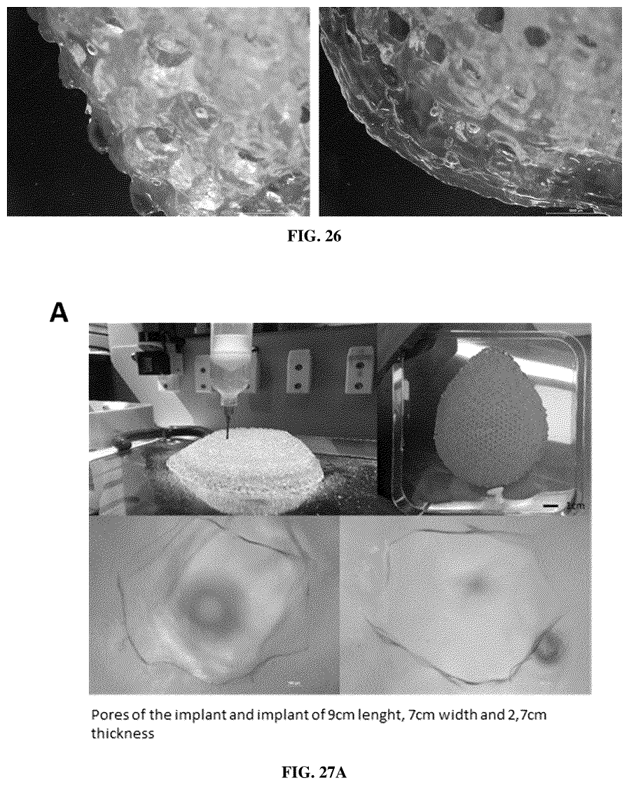

The resulting architecture was studied using a microscope (Olympus, ×4 magnification). The results of these observations are shown in FIG. 26.

The addition of a perimeter to the base of the implant makes it possible to obtain a more cohesive base with fewer asperities at the periphery, thus limiting inflammatory friction in vivo.

### Example 17—Production of Large Volume Porous Implants from Alginate/Gelatin Hydrogel

Implant 1: 200 mL of AG hydrogels were prepared from protocol #1 and then a 12 cm long, 10 cm wide and 5 cm thick anatomical breast-like implant was printed from a variant of protocol #7 (840 μm inner diameter extrusion nozzle and 30 mm/sec printing speed) with a single area of porosity and then cross-linked with protocol #4 (200 mL instead of 100 mL of consolidation solution for a large implant). The average pore size of the resulting implant was measured with an optical microscope and the dimensions of the implant were measured with a caliper. After cross-linking, an implant of 9 cm length, 7 cm width and 2.7 cm thickness is obtained with an average pore size of 1380+/−57 μm.

Implant 2: 500 mL of AG hydrogels were prepared from protocol #1 and then a 7 cm radius, 6 cm thick half-spherical breast-like implant was printed from a variant of protocol #7 (840 μm inner diameter extrusion nozzle and 30 mm/sec printing speed) with a single pore area, and then cross-linked with protocol #4 (700 mL instead of 100 mL of consolidation solution for a large implant). The average pore size of the resulting implant was measured with an optical microscope and the dimensions of the implant were measured with a caliper. After cross-linking, an implant with a diameter of 12.5 cm and a thickness of 5.3 cm is obtained with an average pore size of 3354 μm+/−273 μm.

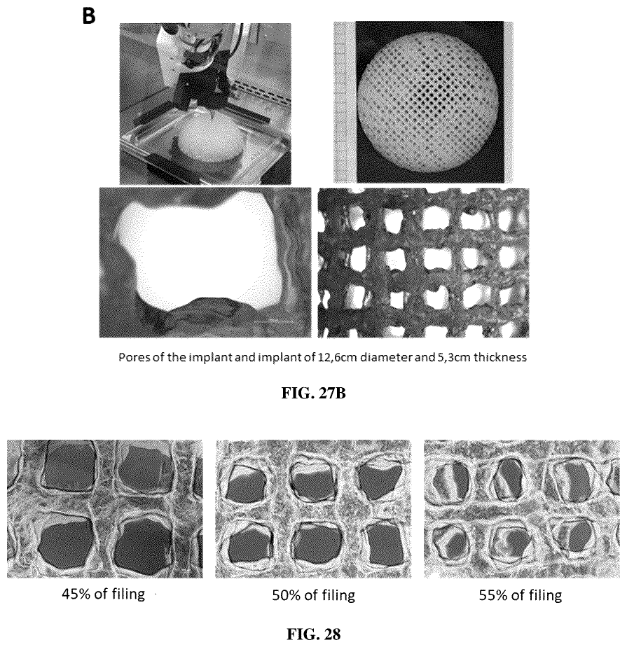

Images of these implants are shown in FIGS. 27A and 27B.

### Example 18—Measurement of the Distribution in Space of the Pores of the Same Zone of an Implant

Semi-anatomical sized breast prosthesis implants (Height: 8.83 cm; Width: 6.37 cm; Height: 2.86 cm) are produced from protocol #1 and protocol #7 and then cross-linked from protocol #4. These implants are produced with different filling rates (45, 50 and 55%).

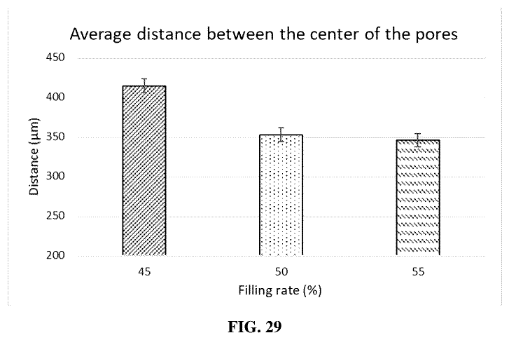

The distances separating the centers of the pores (of square shape) obtained were studied. These dimensions were measured from images taken with a microscope (Olympus, ×4 magnification). The results of these measurements are shown in FIGS. 28 and 29. The distance separating the centers of the pores turns out to be reproducible for each filling rate and varies between the different rates. These observations reflect a homogeneous distribution of pores within a zone of the implant with a defined filling rate.

1.-31. (canceled)

32. A three-dimensional body implant which comprises a hydrogel comprising cross-linked gelatin and cross-linked alginate, wherein said hydrogel has a mechanical strength of from 1 kPa to 1000 kPa and that said implant has at least one porous zone, the porous zone comprising a plurality of pores each having a pore size, the porous zone having an overall porosity of from 100 μm to 10000 μm, the overall porosity corresponding to an average of the pore sizes measured in the porous zone.

33. The implant according to claim 32, wherein the pores of the porous zone have homogeneous pore sizes.

34. The implant according to claim 32, wherein the pores of the porous zone are homogeneously distributed.

35. The implant according to claim 32, wherein the pores of the porous zone extend along central axes having respectively homogeneous orientations.

36. The implant according to claim 32, wherein the pores of the porous zone have respectively homogeneous geometries.

37. The implant according to claim 32, wherein the pores of the porous zone are formed by the three-dimensional structure of the implant in the form of gyroid, cubic or hexagonal lattices.

38. The implant according to claim 32, comprising one porous zone or a plurality of porous zones.

39. The implant according to claim 38, wherein said plurality of porous zones comprises at least two porous zones in which the pores have different pore sizes and/or shapes.

40. The implant according to claim 39, wherein the porous zones are arranged to form a gradient of pore sizes distributed across the implant, the porous zones succeeding each other along a gradient direction in an order selected from an ascending order and a descending order of pore sizes.

41. The implant according to claim 40, wherein the implant comprises:
a first porous zone forming a base representing 5% to 40% of a total volume of the implant, and having a pore size between 500 micrometers and 5000 micrometers,
a second porous zone forming a core representing 20% to 70% of the total volume of the implant and having a pore size between 500 micrometers and 2500 micrometers,
a third porous zone forming a shell representing 5% to 40% of the total volume of the implant, and having a pore size between 1000 micrometers to 10000 micrometers.

42. The implant according to claim 41, wherein the implant comprises:
a first porous zone forming a base representing 20% to 40% of a total volume of the implant, and having a pore size between 500 micrometers and 5000 micrometers,
a second porous zone forming a core representing 30% to 50% of the total volume of the implant and having a pore size between 500 micrometers and 2500 micrometers
a third porous zone forming a shell representing 10% to 40% of the total volume of the implant, and having a pore size between 1000 micrometers to 10000 micrometers.

43. The implant according to claim 32, having at least one non-porous zone, the non-porous zone having a fill rate greater than 99%.

44. The implant according to claim 43, wherein said at least one non-porous zone comprises a perimeter surrounding the porous zone.

45. The implant according to claim 32, wherein said at least one porous zone covers a substantial portion of the implant.

46. The implant according to claim 32, consisting of a plurality of layers each having a mesh made up of a plurality of meshes, the layers being stacked on top of one another in such a way that the meshes form the pores.

47. The implant according to claim 32, wherein the implant has a volume in a range from 0.05 mL to 3 L.

48. The implant according to claim 32, wherein the implant is a breast implant.

49. A manufacturing process for obtaining a three-dimensional body implant comprising successively:
a step of preparing a hydrogel comprising gelatin and alginate,
a step of three-dimensionally shaping the hydrogel so as to form at least one porous zone, the porous zone comprising a plurality of pores each having a pore size, the porous zone having an overall porosity of between 100 μm and 10000 μm, the overall porosity corresponding to an average of the pore sizes measured in the porous zone, and
a step of cross-linking the hydrogel with at least one divalent cation, and transglutaminase, said hydrogel having a mechanical strength of 1 kPa to 1000 kPa.

50. The manufacturing process according to claim 49, wherein the at least one divalent cation is calcium.

51. The manufacturing process according to claim 49, wherein, during the cross-linking step, the divalent cation and the transglutaminase are added concomitantly.

52. The manufacturing process according to claim 49, wherein the hydrogel further comprises cross-linked fibrinogen.

53. The manufacturing process according to claim 49, in which, during the three-dimensional shaping step, an additive manufacturing process is implemented.

54. The manufacturing process according to claim 49, further comprising a sterilization step.

55. A method for implementing the implant according to claim 32 in the context of reconstructive or cosmetic surgery, comprising a step of implanting the implant in the body of a subject.
